(19) United States
(12) Patent Application Publication
(10) Pub. No.: US 2003/0108976 A1
(43) Pub. Date: Jun. 12, 2003

(54) Method and apparatus for improving clinical accuracy of analyte measurements

(76) Inventors: James R. Braig, Piedmont, CA (US); Peter Rule, Los Altos Hills, CA (US); Philip C. Hartstein, Cupertino, CA (US); Heidi S. Smith, Union City, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/268,599

(22) Filed: Oct. 9, 2002

Related U.S. Application Data
(60) Provisional application No. 60/328,072, filed on Oct. 9, 2001.
(60) Provisional application No. 60/339,116, filed on Nov. 7, 2001.

(51) Int. Cl. C12Q 1/54; G01F 1/64; G01N 27/26; G01N 33/50

(52) U.S. Cl. 435/14; 205/777.5

(57) ABSTRACT

Herein is described a system that includes a processing circuit for identifying possible zone D errors among estimated blood glucose concentration values. The system converts estimated blood glucose concentration values which are identified as possible zone D errors into adjusted blood glucose concentration values which are lower in blood glucose concentration magnitude than their corresponding estimated blood glucose concentration values, thereby decreasing the occurrence of zone D errors. Herein is also disclosed a method for improving the clinical accuracy of an analyte concentration measurement. One method includes a first act of computing an estimated analyte concentration having an associated first error that is clinically significant and a second act of processing the estimated analyte concentration to generate an adjusted analyte concentration having a second error that is clinically insignificant.

### REFERENCE TO RELATED APPLICATIONS

This application claims the benefit of U.S. Provisional Application No. 60/328,072, filed Oct. 9, 2001 and U.S. Provisional Application No. 60/339,116, filed Nov. 7, 2001. The above-mentioned patent applications are hereby incorporated by reference in their entirety and made a part of this specification.

### FIELD OF THE INVENTION

This invention relates generally to determining analyte concentrations in material samples.

### DESCRIPTION OF THE RELATED ART

The analysis of materials and the determination of the presence or concentration of chemical species contained therein is a common and important process in chemistry and biology. Particularly important is the analysis of biological fluids, such as blood, urine, or saliva, to determine the concentration of various constituents. Also of great importance is the measurement of the concentration of various chemical constituents embedded within biological materials, such as tissue. Chemical analysis of blood, urine, and other biological fluids is crucial to the diagnosis, management, treatment, and care of a wide variety of diseases and medical conditions. In the case of diabetes, monitoring of blood glucose levels several times a day is necessary to the efficient management of this disease in many patients. Analysis of various blood components is of importance in both the diagnosis and treatment of diseases of the circulatory system. For example, the level of various types of cholesterol in the blood has a strong correlation with the onset of heart disease. Urine analysis provides valuable information relating to kidney function and kidney disease. The concentration of alcohol in the blood is known to be related to a subject's physical response time and coordination and can provide information related to, for example, the individual's fitness to drive a motor vehicle.

### SUMMARY OF THE INVENTION

Instruments that perform analyte analysis are not always accurate. In many cases, the errors are small and do not affect the clinical significance of the measurement. In some cases, however, an erroneous measurement may cause an incorrect course of action. For example, an instrument that erroneously reports that a glucose level is at an appropriate level may cause a patient to fail to administer glucose when it is needed.

The clinical significance of erroneous readings from an instrument can be minimized by applying a transfer function to a raw measurement. For example, in one embodiment the Clarke error grid is employed to illustrate the clinical significance of erroneous glucose level measurements. In this embodiment, erroneous measurements falling into zone A are clinically accurate, erroneous measurements falling into zone B are clinically neutral or benign errors, erroneous measurements falling into zone C result in treating an acceptable glucose level, erroneous measurements falling into zone D result in a failure to treat when a glucose level is unacceptable, and erroneous measurements falling into zone E result in the wrong treatment being given for an unacceptable glucose level. Erroneous measurements falling into zones C, D, and E are clinically significant. Adjusting raw glucose measurements so that the displayed measurements tend to fall into zone A or zone B instead of zones C, D or E avoids clinically significant errors.

Herein is described a system that includes a processing circuit for identifying possible zone D errors among estimated blood glucose concentration values. The system converts estimated blood glucose concentration values which are identified as possible zone D errors into adjusted blood glucose concentration values which are lower in blood glucose concentration magnitude than their corresponding estimated blood glucose concentration values, thereby decreasing the occurrence of zone D errors.

The blood glucose apparatus has an associated error band and the processing circuit identifies the possible zone D errors as estimated blood glucose values which are greater than or equal to a threshold value of clinical significance and less than the sum of the threshold value and the error band. In one embodiment, the error band comprises a maximum expected deviation of the estimated blood glucose concentration values from corresponding actual blood glucose concentration values. In another embodiment, the error band comprises a deviation from identity which encompasses a selected percentage of measurements, such as at least 80 percent of expected deviations from actual blood glucose concentration values.

In one embodiment, the threshold value of clinical significance corresponds to the lowest portion of the border between zone D and zone A of the Clarke error grid. In another embodiment, the threshold value of clinical significance is selected from the range of about 50 mg/dL to about 80 mg/dL. In yet another embodiment, the threshold value of clinical significance is about 70 mg/dL. In another embodiment, the processing circuit converts the estimated blood glucose concentration values which are identified as possible zone D errors, by subtracting the error band from the estimated blood glucose concentration values which are identified as possible zone D errors.

Herein is also described a blood glucose detection system for reducing occurrences of measurement errors that exceed a threshold value of clinical significance. The system has an associated error band, and includes a processor that converts an estimated blood glucose concentration value of at least the threshold value and less than the sum of the threshold value and the error band into an adjusted blood glucose concentration value that is below the border between zones D and A of the Clarke error grid.

In one embodiment, the system has an associated error band and the processing circuit converts the estimated blood glucose concentration values which are identified as possible zone D errors, by subtracting the error band from the estimated blood glucose concentration values which are identified as possible zone D errors. In another embodiment, the threshold value of clinical significance corresponds to the lowest portion of the border between zone D and zone A of the Clarke error grid. In yet another embodiment, the error band comprises a maximum expected deviation from actual blood glucose concentration values. In yet another embodiment, the error band comprises a selected percentage of measurements, such as at least 80 percent of expected deviations from actual blood glucose concentration values.

One embodiment of an analyte detection system includes a processing circuit and a module executable by the processing circuit whereby the processing circuit receives an estimated analyte concentration having an associated first error that is clinically significant, and the processing circuit applies a transfer function to the estimated analyte concentration to generate an adjusted analyte concentration having a second error that is clinically insignificant. In one embodiment, the estimated analyte concentration is an estimate of the concentration of glucose within blood. In one embodiment, the adjusted analyte concentration differs from the estimated analyte concentration where the estimated analyte concentration has a value from about 70 mg/dL to about 85 mg/dL. In another embodiment, the system has an associated error band and the adjusted analyte concentration differs from the estimated analyte concentration where the estimated analyte concentration has a value greater than or equal to a threshold value of clinical significance and less than or equal to the sum of the threshold value and the error band.

In one embodiment, the threshold value of clinical significance is selected from the range of about 50 mg/dL to about 80 mg/dL. In another embodiment, the threshold value of clinical significance is selected from the range of about 60 mg/dL to about 80 mg/dL. In yet another embodiment, the threshold value of clinical significance is selected from the range of about 50 mg/dL to about 70 mg/dL. In a further embodiment, the threshold value of clinical significance is selected from the range of about 60 mg/dL to about 70 mg/dL. In one embodiment, the threshold value of clinical significance corresponds to the lowest portion of a border on the Clarke error grid below which estimated analyte concentrations are zone A errors and above which estimated analyte concentrations are zone D errors.

In one embodiment, the system has an associated error band and the adjusted analyte concentration is about one error band lower than the estimated analyte concentration. In another embodiment, at least a portion of the transfer function comprises an arc having a radius equivalent to the error band and a center point having horizontal and vertical axis coordinates equal to a threshold value of clinical significance. In yet another embodiment, at least a portion of the transfer function comprises an arc. In one embodiment, the transfer function is continuous. In another embodiment, the processor adjusts the estimated analyte concentrations having a value greater than or equal to a threshold value of clinical significance and less than or equal to the sum of the threshold value and the error band, to a substantially uniform adjusted value equal to the threshold value. In yet another embodiment, the system has an associated error band and the processor adjusts the estimated analyte concentrations having a value greater than or equal to a threshold value of clinical significance and less than or equal to the sum of the threshold value and the error band, to a maximum value less than the threshold value.

In one embodiment, the transfer function is selected to correspond to an individual user. In one embodiment, the detected analyte is organic. In another embodiment, the detected analyte is inorganic. In one embodiment, the analyte is detected from whole blood. In one embodiment, a plurality of analytes are detected. In one embodiment, the analyte is detected from tissue. In one embodiment, the analyte is detected from fluid. In one embodiment, the analyte is detected from the group consisting of interstitial fluid, intercellular fluid, and whole blood. In one embodiment, the system is for home use. In another embodiment, the system is for field use.

Herein is also described an apparatus for providing an adjusted analyte concentration, wherein reporting an analyte concentration having a value below a threshold value of clinical significance causes a first course of treatment and reporting an analyte concentration having a value above the threshold value of clinical significance causes a second course of treatment. The apparatus includes a processing circuit that receives an estimated analyte concentration and applies a transfer function to the estimated analyte concentration to provide an adjusted analyte concentration. The adjusted analyte concentration differs from the estimated analyte concentration when the estimated analyte concentration is in the proximity of the threshold value of clinical significance.

In one embodiment, the estimated analyte concentration is an estimate of the concentration of glucose within blood. In one embodiment, the adjusted analyte concentration differs from the estimated analyte concentration where the estimated analyte concentration has a value from about 70 mg/dL to about 85 mg/dL. In one embodiment, estimated analyte concentrations having a value from about 70 mg/dL to about 85 mg/dL are adjusted. In one embodiment, the threshold value of clinical significance is selected from the range of about 50 mg/dL to about 80 mg/dL. In one embodiment, the threshold value of clinical significance is selected from the range of about 60 mg/dL to about 80 mg/dL. In one embodiment, the threshold value of clinical significance is selected from the range of about 50 mg/dL to about 70 mg/dL. In one embodiment, the threshold value of clinical significance is selected from the range of about 60 mg/dL to about 70 mg/dL.

In one embodiment, the apparatus has an associated error band and the adjusted analyte concentration differs from the estimated analyte concentration where the estimated analyte concentration has a value greater than or equal to a threshold value of clinical significance and less than or equal to the sum of the threshold value and the error band. In one embodiment, the threshold value is selected from the range of about 50 mg/dL to about 80 mg/dL. In one embodiment, the error band is in the range of about 10 mg/dL to about 50 mg/dL. In one embodiment, the transfer function is continuous. In one embodiment, the apparatus has an associated error band and the adjusted analyte concentration differs from the estimated analyte concentration by an amount equivalent to the sum of the threshold value and the error band. In one embodiment, the apparatus has an associated error band and the adjusted analyte concentration differs from the estimated analyte concentration by an amount equivalent to the difference between the estimated analyte concentration and the threshold value. In one embodiment, the apparatus has an associated error band and the adjusted analyte concentration is selected as the greater of the threshold value and about 120% of the difference between the estimated analyte concentration and the error band. In one embodiment, the apparatus has an associated error band and at least a portion of the transfer function comprises an arc having a radius equivalent to the error band and a center point having horizontal and vertical axis coordinates equal to a threshold value of clinical significance. In one embodiment, at least a portion of the transfer function comprises an arc segment. In one embodiment, the adjusted analyte concentration is substantially equivalent to the estimated analyte concentration value when the estimated analyte concentration value is not in the proximity of the threshold value of clinical significance. In one embodiment, the transfer function is selected to correspond to an individual user. In one embodiment, the processing circuit reduces a maximum deviation for the estimated analyte concentration.

Herein is also described an apparatus for improving the clinical accuracy of an analyte concentration measurement. The apparatus includes a detection means for obtaining the analyte concentration measurement and a processor means for adjusting the measurement to avoid reporting erroneous measurements that are clinically significant.

Herein is also described a system for determining an analyte concentration. The system includes a processing circuit which computes a first analyte concentration measurement value accurate within a first error band of the system, determines whether the first analyte concentration measurement value is greater than or equal to a threshold value of clinical significance and less than the sum of the threshold value and the first error band, and computes a second analyte concentration measurement value when the first analyte concentration measurement value is greater than or equal to the threshold value of clinical significance and less than the sum of the threshold value and the first error band, wherein the second analyte concentration is accurate within a second error band of the system.

In one embodiment, the processor applies a transfer function to obtain an adjusted analyte concentration measurement value when the second analyte concentration measurement value is greater than or equal to the threshold value of clinical significance and less than the sum of the threshold value and the second error band. In one embodiment, the processor computes the second analyte concentration measurement value by increased sampling of the analyte concentration. In one embodiment, the processor computes the second analyte concentration measurement value by increasing a sampling time period.

Herein is also described a method for improving the clinical accuracy of an analyte concentration measurement. In one embodiment, the method includes a first act of computing an estimated analyte concentration having an associated first error that is clinically significant and a second act of processing the estimated analyte concentration to generate an adjusted analyte concentration having a second error that is clinically insignificant.

In one embodiment, the method further includes determining zones of clinical significance. In one embodiment, the estimated analyte concentration is an estimate of the concentration of glucose within blood. In one embodiment, the adjusted analyte concentration differs from the estimated analyte concentration where the estimated analyte concentration has a value from about 70 mg/dL to about 85 mg/dL. In one embodiment, the method further includes determining an error band, wherein the adjusted analyte concentration differs from the estimated analyte concentration where the estimated analyte concentration has a value greater than or equal to a threshold value of clinical significance and less than or equal to the sum of the threshold value and the error band. In one embodiment, the threshold value of clinical significance is selected from the range of about 50 mg/dL to about 80 mg/dL. In one embodiment, the threshold value of clinical significance is selected from the range of about 60 mg/dL to about 80 mg/dL. In one embodiment, the threshold value of clinical significance is selected from the range of about 50 mg/dL to about 70 mg/dL. In one embodiment, the threshold value of clinical significance is selected from the range of about 60 mg/dL to about 70 mg/dL. In one embodiment, the threshold value of clinical significance corresponds to the lowest portion of a border on the Clarke error grid below which estimated analyte concentrations are zone A errors and above which estimated analyte concentrations are zone D errors.

In one embodiment, the method includes determining an associated error band, wherein the adjusted analyte concentration is about one error band lower than the estimated analyte concentration. In one embodiment, the method includes determining an associated error band, wherein the processing adjusts the estimated analyte concentrations having a value greater than or equal to a threshold value of clinical significance and less than or equal to the sum of the threshold value and the error band, to a substantially uniform adjusted value equal to the threshold value. In one embodiment, the method includes determining an associated error band, wherein the processing adjusts the estimated analyte concentrations having a value greater than or equal to a threshold value of clinical significance and less than or equal to the sum of the threshold value and the error band, to a maximum value less than the threshold value. In one embodiment, the processing is performed using a transfer function.

In one embodiment, the transfer function is derived from a flow chart. In one embodiment, the transfer function is derived from a procedural checklist. In one embodiment, the transfer function is derived from a graph. In one embodiment, the transfer function is derived from a lookup table. In one embodiment, the method includes determining an associated error band, wherein at least a portion of the transfer function comprises an arc having a radius equivalent to the error band and a center point having horizontal and vertical axis coordinates equal to a threshold value of clinical significance. In one embodiment, at least a portion of the transfer function comprises an arc. In one embodiment, the transfer function is continuous. In one embodiment, the transfer function is selected to correspond to an individual user.

In one embodiment, the processing is performed using a filter. In one embodiment, the estimated analyte concentration is a filter input. In one embodiment, the adjusted analyte concentration provides feedback to the filter. In one embodiment, the filter is digital. In one embodiment, a sample period is less than one second. In one embodiment, a sample period is between one second and one minute. In one embodiment, a sample period is between one minute and one hour. In one embodiment, a sample period is greater than one hour.

In one embodiment, the detected analyte is organic. In one embodiment, the detected analyte is inorganic. In one embodiment, the analyte is detected from whole blood. In one embodiment, a plurality of analytes are detected. In one embodiment, the analyte is detected from tissue. In one embodiment, the analyte is detected from fluid. In one embodiment, the analyte is detected from the group consisting of interstitial fluid, intercellular fluid, and whole blood.

### BRIEF DESCRIPTION OF THE DRAWINGS

FIG. 1 is a schematic view of a noninvasive optical detection system.

FIG. 2 is a perspective view of a window assembly for use with the noninvasive detection system.

FIG. 3 is an exploded schematic view of an alternative window assembly for use with the noninvasive detection system.

FIG. 4 is a plan view of the window assembly connected to a cooling system.

FIG. 5 is a plan view of the window assembly connected to a cold reservoir.

FIG. 6 is a cutaway view of a heat sink for use with the noninvasive detection system.

FIG. 6A is a cutaway perspective view of a lower portion of the noninvasive detection system of FIG. 1.

FIG. 7 is a schematic view of a control system for use with the noninvasive optical detection system.

FIG. 8 depicts a first methodology for determining the concentration of an analyte of interest.

FIG. 9 depicts a second methodology for determining the concentration of an analyte of interest.

FIG. 10 depicts a third methodology for determining the concentration of an analyte of interest.

FIG. 11 depicts a fourth methodology for determining the concentration of an analyte of interest.

FIG. 12 depicts a fifth methodology for determining the concentration of an analyte of interest.

FIG. 13 is a schematic view of a reagentless whole-blood detection system.

FIG. 14 is a perspective view of one embodiment of a cuvette for use with the reagentless whole-blood detection system.

FIG. 15 is a plan view of another embodiment of a cuvette for use with the reagentless whole-blood detection system.

FIG. 16 is a disassembled plan view of the cuvette shown in FIG. 15.

FIG. 16A is an exploded perspective view of the cuvette of FIG. 15.

FIG. 17 is a side view of the cuvette of FIG. 15.

FIG. 18 depicts the classification of erroneous measurements by their clinical implications.

FIG. 19 depicts the classification of erroneous measurements by their clinical implications.

FIG. 20 depicts the classification of erroneous measurements by their clinical implications.

FIG. 21 depicts the classification of erroneous measurements by their clinical implications for an instrument having a given error band.

FIG. 22 depicts possible actual glucose concentrations given a raw measurement.

FIG. 23 depicts simulated measurements from a theoretical instrument having a maximum deviation of ±30 mg/dL.

FIG. 24 depicts simulated measurements from a theoretical instrument having a maximum deviation of ±15 mg/dL.

FIG. 25 depicts additional simulated measurements from a theoretical instrument having a maximum deviation of ±15 mg/dL.

FIG. 26 depicts a representation of a transfer function.

FIG. 27 depicts simulated measurements from a theoretical instrument having a maximum deviation of ±20 mg/dL.

FIG. 28 depicts simulated measurements that are adjusted to avoid clinically significant false readings.

FIG. 29 depicts a representation of a transfer function.

FIG. 30 depicts a representation of a transfer function.

FIG. 31 depicts a representation of a transfer function.

FIG. 32 depicts a representation of a transfer function.

FIG. 33 depicts a representation of a transfer function.

FIG. 34 depicts representations of transfer functions.

### DETAILED DESCRIPTION OF THE PREFERRED EMBODIMENTS

Instruments that perform analyte analysis are not always accurate. In many cases, the errors are small and do not affect the clinical significance of the measurement. In some cases, however, an erroneous measurement may cause an incorrect course of action. For example, an instrument that erroneously reports that a glucose level is at an appropriate level may cause a patient to fail to administer glucose when it is needed.

The clinical significance of erroneous readings from an instrument can be minimized by applying a transfer function to a raw measurement. For example, in one embodiment the Clarke error grid is employed to illustrate the clinical significance of erroneous glucose level measurements. In this embodiment, erroneous measurements falling into zone A are clinically accurate, erroneous measurements falling into zone B are clinically neutral or benign errors, erroneous measurements falling into zone C result in treating an acceptable glucose level, erroneous measurements falling into zone D result in a failure to treat when a glucose level is unacceptable, and erroneous measurements falling into zone E result in the wrong treatment being given for an unacceptable glucose level. Erroneous measurements falling into zones C, D, and E are clinically significant. Adjusting raw glucose measurements so that the displayed measurements tend to fall into zone A or zone B instead of zones C, D or E avoids clinically significant, errors.

Generally, “clinically significant” as used herein is a broad term that is used in its ordinary sense and refers, without limitation, to that which causes an adverse impact on clinical decision making. A clinically significant error may result in a patient failing to receive treatment when treatment is needed, receiving treatment when treatment is not needed, or receiving a wrong treatment. Accordingly, in one embodiment, clinically significant errors may comprise errors falling into zones C, D, and E of the Clarke error grid. In another embodiment, clinically significant errors may comprise errors falling into zones D and E of the Clarke error grid.

Herein is disclosed a method and apparatus for improving the clinical accuracy of an analyte concentration measurement. Part I describes the measurement of an analyte by using systems such as a non-invasive analyte detection system or a whole blood analyte detection system. In a presently preferred embodiment, the analyte concentration measurement system measures the concentration of glucose in blood. An analyte concentration measurement is clinically accurate when the measurement results in correct treatment of a patient. However, an instrument may erroneously report a measurement that causes a patient to receive the wrong treatment. Part II describes a method and apparatus for improving the clinical accuracy of an analyte concentration measurement. In a presently preferred embodiment, the system uses the Clarke error grid to determine which analyte concentration measurements have the potential to cause clinically significant errors. These measurements are then adjusted to avoid reporting a clinically erroneous result.

Although certain preferred embodiments and examples are disclosed below, it will be understood by those skilled in the art that the invention extends beyond the specifically disclosed embodiments to other alternative embodiments and/or uses of the invention and obvious modifications and equivalents thereof. Thus, it is intended that the scope of the invention herein disclosed should not be limited by the particular disclosed embodiments described below.

### I. OVERVIEW OF ANALYTE DETECTION SYSTEMS

Disclosed herein are analyte detection systems, including a noninvasive system discussed largely in part A below and a whole-blood system discussed largely in part B below. Also disclosed are various methods, including methods for detecting the concentration of an analyte in a material sample. Both the noninvasive system/method and the whole-blood system/method can employ optical measurement. As used herein with reference to measurement apparatus and methods, “optical” is a broad term and is used in its ordinary sense and refers, without limitation, to identification of the presence or concentration of an analyte in a material sample without requiring a chemical reaction to take place. As discussed in more detail below, the two approaches each can operate independently to perform an optical analysis of a material sample. The two approaches can also be combined in an apparatus, or the two approaches can be used together to perform different steps of a method.

In one embodiment, the two approaches are combined to perform calibration of an apparatus, e.g., of an apparatus that employs a noninvasive approach. In another embodiment, an advantageous combination of the two approaches performs an invasive measurement to achieve greater accuracy and a whole-blood measurement to minimize discomfort to the patient. For example, the whole-blood technique may be more accurate than the noninvasive technique at certain times of the day, e.g., at certain times after a meal has been consumed, or after a drug has been administered.

It should be understood, however, that any of the disclosed devices may be operated in accordance with any suitable detection methodology, and that any disclosed method may be employed in the operation of any suitable device. Furthermore, the disclosed devices and methods are applicable in a wide variety of situations or modes of operation, including but not limited to invasive, noninvasive, intermittent or continuous measurement, subcutaneous implantation, wearable detection systems, or any combination thereof.

Any method which is described and illustrated herein is not limited to the exact sequence of acts described, nor is it necessarily limited to the practice of all of the acts set forth. Other sequences of events or acts, or less than all of the events, or simultaneous occurrence of the events, may be utilized in practicing the method(s) in question.

### A. Noninvasive System

1. Monitor Structure

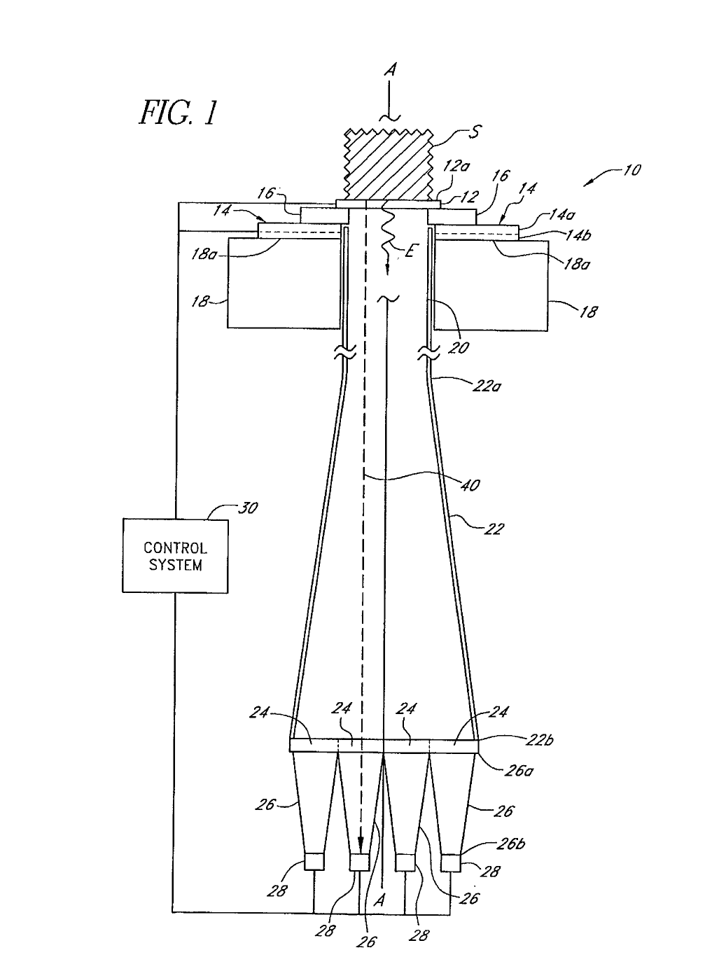

FIG. 1 depicts a noninvasive optical detection system (hereinafter “noninvasive system”) 10 in a presently preferred configuration. The depicted noninvasive system 10 is particularly suited for noninvasively detecting the concentration of an analyte in a material sample S, by observing the infrared energy emitted by the sample, as will be discussed in further detail below.

As used herein, the term “noninvasive” is a broad term and is used in its ordinary sense and refers, without limitation, to analyte detection devices and methods which have the capability to determine the concentration of an analyte in in-vivo tissue samples or bodily fluids. It should be understood, however, that the noninvasive system 10 disclosed herein is not limited to noninvasive use, as the noninvasive system 10 may be employed to analyze an in-vitro fluid or tissue sample which has been obtained invasively or noninvasively. As used herein, the term “invasive” (or, alternatively, “traditional”) is a broad term and is used in its ordinary sense and refers, without limitation, to analyte detection methods which involve the removal of fluid samples through the skin. As used herein, the term “material sample” is a broad term and is used in its ordinary sense and refers, without limitation, to any collection of material which is suitable for analysis by the noninvasive system 10. For example, the material sample S may comprise a tissue sample, such as a human forearm, placed against the noninvasive system 10. The material sample S may also comprise a volume of a bodily fluid, such as whole blood, blood component(s), interstitial fluid or intercellular fluid obtained invasively, or saliva or urine obtained noninvasively, or any collection of organic or inorganic material. As used herein, the term “analyte” is a broad term and is used in its ordinary sense and refers, without limitation, to any chemical species the presence or concentration of which is sought in the material sample S by the noninvasive system 10. For example, the analyte(s) which may be detected by the noninvasive system 10 include but not are limited to glucose, ethanol, insulin, water, carbon dioxide, blood oxygen, cholesterol, bilirubin, ketones, fatty acids, lipoproteins, albumin, urea, creatinine, white blood cells, red blood cells, hemoglobin, oxygenated hemoglobin, carboxyhemoglobin, organic molecules, inorganic molecules, pharmaceuticals, cytochrome, various proteins and chromophores, microcalcifications, electrolytes, sodium, potassium, chloride, bicarbonate, and hormones. As used herein to describe measurement techniques, the term “continuous” is a broad term and is used in its ordinary sense and refers, without limitation, to the taking of discrete measurements more frequently than about once every 10 minutes, and/or the taking of a stream or series of measurements or other data over any suitable time interval, for example, over an interval of one to several seconds, minutes, hours, days, or longer. As used herein to describe measurement techniques, the term “intermittent” is a broad term and is used in its ordinary sense and refers, without limitation, to the taking of measurements less frequently than about once every 10 minutes.

The noninvasive system 10 preferably comprises a window assembly 12, although in some embodiments the window assembly 12 may be omitted. One function of the window assembly 12 is to permit infrared energy E to enter the noninvasive system 10 from the sample S when it is placed against an upper surface 12a of the window assembly 12. The window assembly 12 includes a heater layer (see discussion below) which is employed to heat the material sample S and stimulate emission of infrared energy therefrom. A cooling system 14, preferably comprising a Peltier-type thermoelectric device, is in thermally conductive relation to the window assembly 12 so that the temperature of the window assembly 12 and the material sample S can be manipulated in accordance with a detection methodology discussed in greater detail below. The cooling system 14 includes a cold surface 14a which is in thermally conductive relation to a cold reservoir 16 and the window assembly 12, and a hot surface 14b which is in thermally conductive relation to a heat sink 18.

As the infrared energy E enters the noninvasive system 10, it first passes through the window assembly 12, then through an optical mixer 20, and then through a collimator 22. The optical mixer 20 preferably comprises a light pipe having highly reflective inner surfaces which randomize the directionality of the infrared energy E as it passes therethrough and reflects against the mixer walls. The collimator 22 also comprises a light pipe having highly-reflective inner walls, but the walls diverge as they extend away from the mixer 20. The divergent walls cause the infrared energy E to tend to straighten as it advances toward the wider end of the collimator 22, due to the angle of incidence of the infrared energy when reflecting against the collimator walls.

From the collimator 22 the infrared energy E passes through an array of filters 24, each of which allows only a selected wavelength or band of wavelengths to pass therethrough. These wavelengths/bands are selected to highlight or isolate the absorptive effects of the analyte of interest in the detection methodology discussed in greater detail below. Each filter 24 is preferably in optical communication with a concentrator 26 and an infrared detector 28. The concentrators 26 have highly reflective, converging inner walls which concentrate the infrared energy as it advances toward the detectors 28, increasing the density of the energy incident upon the detectors 28.

The detectors 28 are in electrical communication with a control system 30 which receives electrical signals from the detectors 28 and computes the concentration of the analyte in the sample S. The control system 30 is also in electrical communication with the window 12 and cooling system 14, so as to monitor the temperature of the window 12 and/or cooling system 14 and control the delivery of electrical power to the window 12 and cooling system 14.

a. Window Assembly

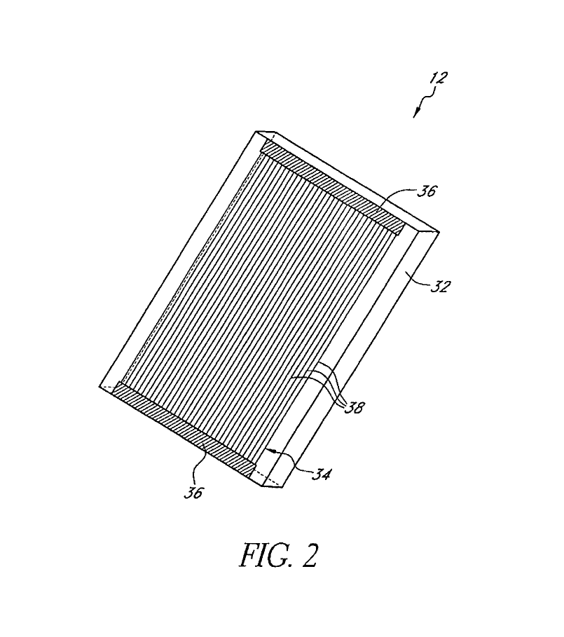

A preferred configuration of the window assembly 12 is shown in perspective, as viewed from its underside (in other words, the side of the window assembly 12 opposite the sample S), in FIG. 2. The window assembly 12 generally comprises a main layer 32 formed of a highly infrared-transmissive material and a heater layer 34 affixed to the underside of the main layer 32. The main layer 32 is preferably formed from diamond, most preferably from chemical-vapor-deposited (“CVD”) diamond, with a preferred thickness of about 0.25 millimeters. In other embodiments alternative materials which are highly infrared-transmissive, such as silicon or germanium, may be used in forming the main layer 32.

The heater layer 34 preferably comprises bus bars 36 located at opposing ends of an array of heater elements 38. The bus bars 36 are in electrical communication with the elements 38 so that, upon connection of the bus bars 36 to a suitable electrical power source (not shown) a current may be passed through the elements 38 to generate heat in the window assembly 12. The heater layer 34 may also include one or more temperature sensors (not shown), such as thermistors or resistance temperature devices (RTDs), to measure the temperature of the window assembly 12 and provide temperature feedback to the control system 30 (see FIG. 1).

Still referring to FIG. 2, the heater layer 34 preferably comprises a first adhesion layer of gold or platinum (hereinafter referred to as the “gold” layer) deposited over an alloy layer which is applied to the main layer 32. The alloy layer comprises a material suitable for implementation of the heater layer 34, such as, by way of example, 10/90 titanium/tungsten, titanium/platinum, nickel/chromium, or other similar material. The gold layer preferably has a thickness of about 4000 Å, and the alloy layer preferably has a thickness ranging between about 300 Å and about 500 Å. The gold layer and/or the alloy layer may be deposited onto the main layer 32 by chemical deposition including, but not necessarily limited to, vapor deposition, liquid deposition, plating, laminating, casting, sintering, or other forming or deposition methodologies well known to those or ordinary skill in the art. If desired, the heater layer 34 may be covered with an electrically insulating coating which also enhances adhesion to the main layer 32. One preferred coating material is aluminum oxide. Other acceptable materials include, but are not limited to, titanium dioxide or zinc selenide.

The heater layer 34 may incorporate a variable pitch distance between centerlines of adjacent heater elements 38 to maintain a constant power density, and promote a uniform temperature, across the entire layer 34. Where a constant pitch distance is employed, the preferred distance is at least about 50-100 microns. Although the heater elements 38 generally have a preferred width of about 25 microns, their width may also be varied as needed for the same reasons stated above.

Alternative structures suitable for use as the heater layer 34 include, but are not limited to, thermoelectric heaters, radiofrequency (RF) heaters, infrared radiation heaters, optical heaters, heat exchangers, electrical resistance heating grids, wire bridge heating grids, or laser heaters. Whichever type of heater layer is employed, it is preferred that the heater layer obscures about 10% or less of the window assembly 12.

In a preferred embodiment, the window assembly 12 comprises substantially only the main layer 32 and the heater layer 34. Thus, when installed in an optical detection system such as the noninvasive system 10 shown in FIG. 1, the window assembly 12 will facilitate a minimally obstructed optical path between a (preferably flat) upper surface 12a of the window assembly 12 and the infrared detectors 28 of the noninvasive system 10. The optical path 32 in the preferred noninvasive system 10 proceeds only through the main layer 32 and heater layer 34 of the window assembly 12 (including any antireflective, index-matching, electrical insulating or protective coatings applied thereto or placed therein), through the optical mixer 20 and collimator 22 and to the detectors 28.

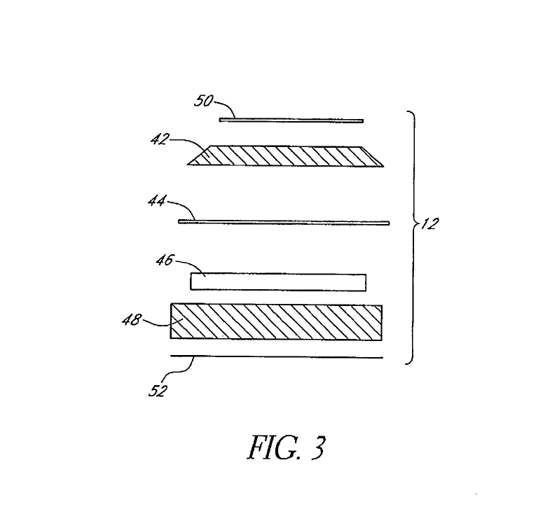

FIG. 3 depicts an exploded side view of an alternative configuration for the window assembly 12, which may be used in place of the configuration shown in FIG. 2. The window assembly 12 depicted in FIG. 3 includes near its upper surface (the surface intended for contact with the sample S) a highly infrared-transmissive, thermally conductive spreader layer 42. Underlying the spreader layer 42 is a heater layer 44. A thin electrically insulating layer (not shown), such as layer of aluminum oxide, titanium dioxide or zinc selenide, may be disposed between the heater layer 44 and the spreader layer 42. (An aluminum oxide layer also increases adhesion of the heater layer 44 to the spreader layer 42.) Adjacent to the heater layer 44 is a thermal insulating and impedance matching layer 46. Adjacent to the thermal insulating layer 46 is a thermally conductive inner layer 48. The spreader layer 42 is coated on its top surface with a thin layer of protective coating 50. The bottom surface of the inner layer 48 is coated with a thin overcoat layer 52. Preferably, the protective coating 50 and the overcoat layer 52 have antireflective properties.

The spreader layer 42 is preferably formed of a highly infrared-transmissive material having a high thermal conductivity sufficient to facilitate heat transfer from the heater layer 44 uniformly into the material sample S when it is placed against the window assembly 12. Other effective materials include, but are not limited to, CVD diamond, diamondlike carbon, gallium arsenide, germanium, and other infrared-transmissive materials having sufficiently high thermal conductivity. Preferred dimensions for the spreader layer 42 are about one inch in diameter and about 0.010 inch thick. As shown in FIG. 3, a preferred embodiment of the spreader layer 42 incorporates a beveled edge. Although not required, an approximate 45-degree bevel is preferred.

The protective layer 50 is intended to protect the top surface of the spreader layer 42 from damage. Ideally, the protective layer is highly infrared-transmissive and highly resistant to mechanical damage, such as scratching or abrasion. It is also preferred that the protective layer 50 and the overcoat layer 52 have high thermal conductivity and antireflective and/or index-matching properties. A satisfactory material for use as the protective layer 50 and the overcoat layer 52 is the multi-layer Broad Band Anti-Reflective Coating produced by Deposition Research Laboratories, Inc. of St. Charles, Mo. Diamondlike carbon coatings are also suitable.

Except as noted below, the heater layer 44 is generally similar to the heater layer 34 employed in the window assembly shown in FIG. 2. Alternatively, the heater layer 44 may comprise a doped infrared-transmissive material, such as a doped silicon layer, with regions of higher and lower resistivity. The heater layer 44 preferably has a resistance of about 2 ohms and has a preferred thickness of about 1,500 angstroms. A preferred material for forming the heater layer 44 is a gold alloy, but other acceptable materials include, but are not limited to, platinum, titanium, tungsten, copper, and nickel.

The thermal insulating layer 46 prevents the dissipation of heat from the heater element 44 while allowing the cooling system 14 to effectively cool the material sample S (see FIG. 1). This layer 46 comprises a material having thermally insulative (e.g., lower thermal conductivity than the spreader layer 42) and infrared transmissive qualities. A preferred material is a germanium-arsenic-selenium compound of the calcogenide glass family known as AMTIR-1 produced by Amorphous Materials, Inc. of Garland, Tex. The pictured embodiment has a diameter of about 0.85 inches and a preferred thickness in the range of about 0.005 to about 0.010 inches. As heat generated by the heater layer 44 passes through the spreader layer 42 into the material sample S, the thermal insulating layer 46 insulates this heat.

The inner layer 48 is formed of thermally conductive material, preferably crystalline silicon formed using a conventional floatzone crystal growth method. The purpose of the inner layer 48 is to serve as a cold-conducting mechanical base for the entire layered window assembly.

The overall optical transmission of the window assembly 12 shown in FIG. 3 is preferably at least 70%. The window assembly 12 of FIG. 3 is preferably held together and secured to the noninvasive system 10 by a holding bracket (not shown). The bracket is preferably formed of a glass-filled plastic, for example Ultem 2300, manufactured by General Electric. Ultem 2300 has low thermal conductivity which prevents heat transfer from the layered window assembly 12.

b. Cooling System

The cooling system 14 (see FIG. 1) preferably comprises a Peltier-type thermoelectric device. Thus, the application of an electrical current to the preferred cooling system 14 causes the cold surface 14a to cool and causes the opposing hot surface 14b to heat up. The cooling system 14 cools the window assembly 12 via the situation of the window assembly 12 in thermally conductive relation to the cold surface 14a of the cooling system 14. It is contemplated that the cooling system 14, the heater layer 34, or both, can be operated to induce a desired time-varying temperature in the window assembly 12 to create an oscillating thermal gradient in the sample S, in accordance with various analyte-detection methodologies discussed herein.

Preferably, the cold reservoir 16 is positioned between the cooling system 14 and the window assembly 12, and functions as a thermal conductor between the system 14 and the window assembly 12. The cold reservoir 16 is formed from a suitable thermally conductive material, preferably brass. Alternatively, the window assembly 12 can be situated in direct contact with the cold surface 14a of the cooling system 14.

In alternative embodiments, the cooling system 14 may comprise a heat exchanger through which a coolant, such as air, nitrogen or chilled water, is pumped, or a passive conduction cooler such as a heat sink. As a further alternative, a gas coolant such as nitrogen may be circulated through the interior of the noninvasive system 10 so as to contact the underside of the window assembly 12 (see FIG. 1) and conduct heat therefrom.

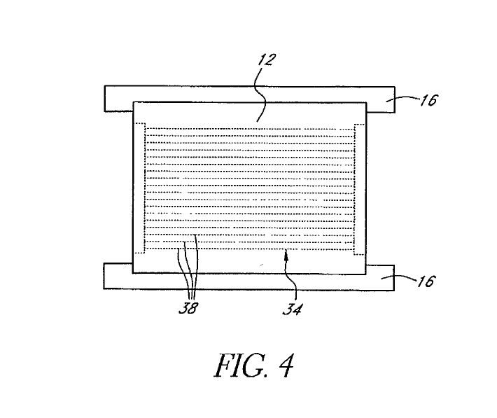

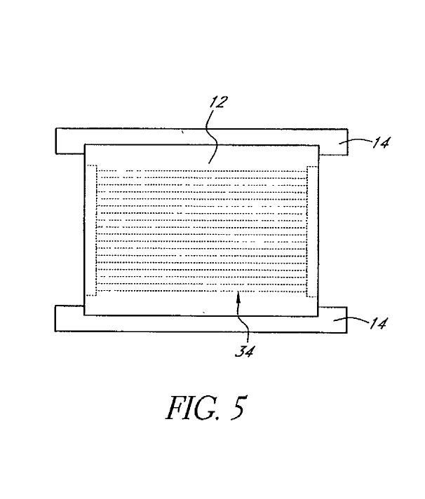

FIG. 4 is a top schematic view of a preferred arrangement of the window assembly 12 (of the type shown in FIG. 2) and the cold reservoir 16, and FIG. 5 is a top schematic view of an alternative arrangement in which the window assembly 12 directly contacts the cooling system 14. The cold reservoir 16/cooling system 14 preferably contacts the underside of the window assembly 12 along opposing edges thereof, on either side of the heater layer 34. With thermal conductivity thus established between the window assembly 12 and the cooling system 14, the window assembly can be cooled as needed during operation of the noninvasive system 10. In order to promote a substantially uniform or isothermal temperature profile over the upper surface of the window assembly 12, the pitch distance between centerlines of adjacent heater elements 38 may be made smaller (thereby increasing the density of heater elements 38) near the region(s) of contact between the window assembly 12 and the cold reservoir 16/cooling system 14. As a supplement or alternative, the heater elements 38 themselves may be made wider near these regions of contact. As used herein, “isothermal” is a broad term and is used in its ordinary sense and refers, without limitation, to a condition in which, at a given point in time, the temperature of the window assembly 12 or other structure is substantially uniform across a surface intended for placement in thermally conductive relation to the material sample S. Thus, although the temperature of the structure or surface may fluctuate over time, at any given point in time the structure or surface may nonetheless be isothermal.

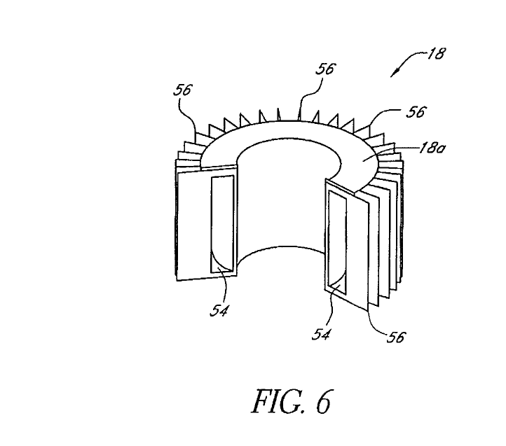

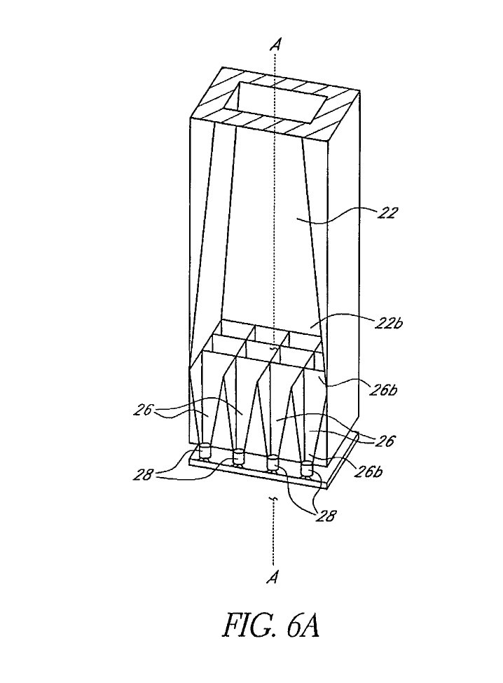

The heat sink 18 drains waste heat from the hot surface 14b of the cooling system 16 and stabilizes the operational temperature of the noninvasive system 10. The preferred heat sink 18 (see FIG. 6) comprises a hollow structure formed from brass or any other suitable material having a relatively high specific heat and high heat conductivity. The heat sink 18 has a conduction surface 18a which, when the heat sink 18 is installed in the noninvasive system 18, is in thermally conductive relation to the hot surface 14b of the cooling system 14 (see FIG. 1). A cavity 54 is formed in the heat sink 18 and preferably contains a phase-change material (not shown) to increase the capacity of the sink 18. A preferred phase change material is a hydrated salt, such as calciumchloride hexahydrate, available under the name TH29 from PCM Thermal Solutions, Inc., of Naperville, Ill. Alternatively, the cavity 54 may be omitted to create a heat sink 18 comprising a solid, unitary mass. The heat sink 18 also forms a number of fins 56 to further increase the conduction of heat from the sink 18 to surrounding air.

Alternatively, the heat sink 18 may be formed integrally with the optical mixer 20 and/or the collimator 22 as a unitary mass of rigid, heat-conductive material such as brass or aluminum. In such a heat sink, the mixer 20 and/or collimator 22 extend axially through the heat sink 18, and the heat sink defines the inner walls of the mixer 20 and/or collimator 22. These inner walls are coated and/or polished to have appropriate reflectivity and nonabsorbance in infrared wavelengths as will be further described below. Where such a unitary heat sink-mixer-collimator is employed, it is desirable to thermally insulate the detector array from the heat sink.

It should be understood that any suitable structure may be employed to heat and/or cool the material sample S, instead of or in addition to the window assembly 12/cooling system 14 disclosed above, so long a proper degree of cycled heating and/or cooling are imparted to the material sample S. In addition other forms of energy, such as but not limited to light, radiation, chemically induced heat, friction and vibration, may be employed to heat the material sample S. It will be further appreciated that heating of the sample can achieved by any suitable method, such as convection, conduction, radiation, etc.

c. Optics

As shown in FIG. 1, the optical mixer 20 comprises a light pipe with an inner surface coating which is highly reflective and minimally absorptive in infrared wavelengths, preferably a polished gold coating, although other suitable coatings may be used where other wavelengths of electromagnetic radiation are employed. The pipe itself may be fabricated from a another rigid material such as aluminum or stainless steel, as long as the inner surfaces are coated or otherwise treated to be highly reflective. Preferably, the optical mixer 20 has a rectangular cross-section (as taken orthogonal to the longitudinal axis A-A of the mixer 20 and the collimator 22), although other cross-sectional shapes, such as other polygonal shapes or circular or elliptical shapes, may be employed in alternative embodiments. The inner walls of the optical mixer 20 are substantially parallel to the longitudinal axis A-A of the mixer 20 and the collimator 22. The highly reflective and substantially parallel inner walls of the mixer 20 maximize the number of times the infrared energy E will be reflected between the walls of the mixer 20, thoroughly mixing the infrared energy E as it propagates through the mixer 20. In a presently preferred embodiment, the mixer 20 is about 1.2 inches to 2.4 inches in length and its cross-section is a rectangle of about 0.4 inches by about 0.6 inches. Of course, other dimensions may be employed in constructing the mixer 20. In particular it is be advantageous to miniaturize the mixer or otherwise make it as small as possible

Still referring to FIG. 1, the collimator 22 comprises a tube with an inner surface coating which is highly reflective and minimally absorptive in infrared wavelengths, preferably a polished gold coating. The tube itself may be fabricated from a another rigid material such as aluminum, nickel or stainless steel, as long as the inner surfaces are coated or otherwise treated to be highly reflective. Preferably, the collimator 22 has a rectangular cross-section, although other cross-sectional shapes, such as other polygonal shapes or circular, parabolic or elliptical shapes, may be employed in alternative embodiments. The inner walls of the collimator 22 diverge as they extend away from the mixer 20. Preferably, the inner walls of the collimator 22 are substantially straight and form an angle of about 7 degrees with respect to the longitudinal axis A-A. The collimator 22 aligns the infrared energy E to propagate in a direction that is generally parallel to the longitudinal axis A-A of the mixer 20 and the collimator 22, so that the infrared energy E will strike the surface of the filters 24 at an angle as close to 90 degrees as possible.

In a presently preferred embodiment, the collimator is about 7.5 inches in length. At its narrow end 22a, the cross-section of the collimator 22 is a rectangle of about 0.4 inches by 0.6 inches. At its wide end 22b, the collimator 22 has a rectangular cross-section of about 1.8 inches by 2.6 inches. Preferably, the collimator 22 aligns the infrared energy E to an angle of incidence (with respect to the longitudinal axis A-A) of about 0-15 degrees before the energy E impinges upon the filters 24. Of course, other dimensions or incidence angles may be employed in constructing and operating the collimator 22.

With further reference to FIGS. 1 and 6A, each concentrator 26 comprises a tapered surface oriented such that its wide end 26a is adapted to receive the infrared energy exiting the corresponding filter 24, and such that its narrow end 26b is adjacent to the corresponding detector 28. The inward-facing surfaces of the concentrators 26 have an inner surface coating which is highly reflective and minimally absorptive in infrared wavelengths, preferably a polished gold coating. The concentrators 26 themselves may be fabricated from a another rigid material such as aluminum, nickel or stainless steel, so long as their inner surfaces are coated or otherwise treated to be highly reflective.

Preferably, the concentrators 26 have a rectangular cross-section (as taken orthogonal to the longitudinal axis A-A), although other cross-sectional shapes, such as other polygonal shapes or circular, parabolic or elliptical shapes, may be employed in alternative embodiments. The inner walls of the concentrators converge as they extend toward the narrow end 26b. Preferably, the inner walls of the collimators 26 are substantially straight and form an angle of about 8 degrees with respect to the longitudinal axis A-A. Such a configuration is adapted to concentrate infrared energy as it passes through the concentrators 26 from the wide end 26a to the narrow end 26b, before reaching the detectors 28.

In a presently preferred embodiment, each concentrator 26 is about 1.5 inches in length. At the wide end 26a, the cross-section of each concentrator 26 is a rectangle of about 0.6 inches by 0.57 inches. At the narrow end 26b, each concentrator 26 has a rectangular cross-section of about 0.177 inches by 0.177 inches. Of course, other dimensions or incidence angles may be employed in constructing the concentrators 26.

d. Filters

The filters 24 preferably comprise standard interference-type infrared filters, widely available from manufacturers such as Optical Coating Laboratory, Inc. (“OCLI”) of Santa Rosa, Calif. In the embodiment illustrated in FIG. 1, a 3×4 array of filters 24 is positioned above a 3×4 array of detectors 28 and concentrators 26. As employed in this embodiment, the filters 24 are arranged in four groups of three filters having the same wavelength sensitivity. These four groups have bandpass center wavelengths of 7.15 [symbol] m±0.03 [symbol] m, 8.40 [symbol] m±0.03 [symbol] m, 9.48 [symbol] m±0.04 [symbol] m, and 11.10 [symbol] m±0.04 [symbol] m, respectively, which correspond to wavelengths around which water and glucose absorb electromagnetic radiation. Typical bandwidths for these filters range from 0.20 [symbol] m to 0.50 [symbol] m.

In an alternative embodiment, the array of wavelength-specific filters 24 may be replaced with a single Fabry-Perot interferometer, which can provide wavelength sensitivity which varies as a sample of infrared energy is taken from the material sample S. Thus, this embodiment permits the use of only one detector 28, the output signal of which varies in wavelength specificity over time. The output signal can be de-multiplexed based on the wavelength sensitivities induced by the Fabry-Perot interferometer, to provide a multiple-wavelength profile of the infrared energy emitted by the material sample S. In this embodiment, the optical mixer 20 may be omitted, as only one detector 28 need be employed.

In still other embodiments, the array of filters 24 may comprise a filter wheel that rotates different filters with varying wavelength sensitivities over a single detector 24. Alternatively, an electronically tunable infrared filter may be employed in a manner similar to the Fabry-Perot interferometer discussed above, to provide wavelength sensitivity which varies during the detection process. In either of these embodiments, the optical mixer 20 may be omitted, as only one detector 28 need be employed.

e. Detectors

The detectors 28 may comprise any detector type suitable for sensing infrared energy, preferably in the mid-infrared wavelengths. For example, the detectors 28 may comprise mercury-cadmium-telluride (MCT) detectors. A detector such as a Fermionics (Simi Valley, Calif.) model PV-9.1 with a PVA481-1 pre-amplifier is acceptable. Similar units from other manufacturers such as Graseby (Tampa, Fla.) can be substituted. Other suitable components for use as the detectors 28 include pyroelectric detectors, thermopiles, bolometers, silicon microbolometers and lead-salt focal plane arrays.

f. Control System

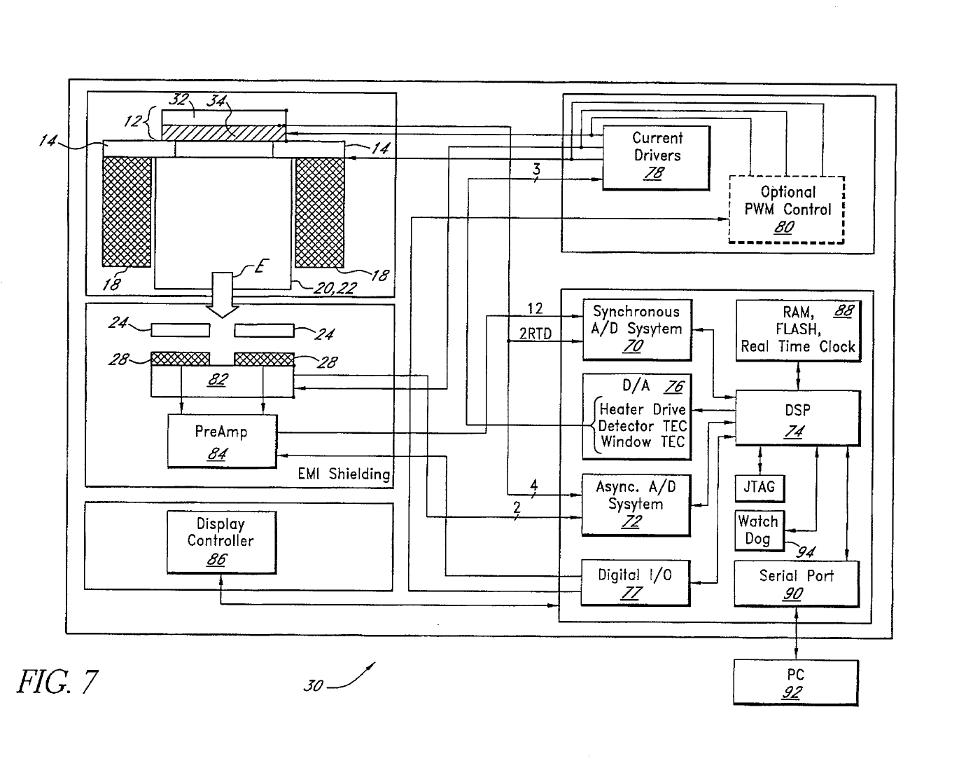

FIG. 7 depicts the control system 30 in greater detail, as well as the interconnections between the control system and other relevant portions of the noninvasive system. The control system includes a temperature control subsystem and a data acquisition subsystem.

In the temperature control subsystem, temperature sensors (such as RTDs and/or thermistors) located in the window assembly 12 provide a window temperature signal to a synchronous analog-to-digital conversion system 70 and an asynchronous analog-to-digital conversion system 72. The A/D systems 70, 72 in turn provide a digital window temperature signal to a digital signal processor (DSP) 74. The processor 74 executes a window temperature control algorithm and determines appropriate control inputs for the heater layer 34 of the window assembly 12 and/or for the cooling system 14, based on the information contained in the window temperature signal. The processor 74 outputs one or more digital control signals to a digital-to-analog conversion system 76 which in turn provides one or more analog control signals to current drivers 78. In response to the control signal(s), the current drivers 78 regulate the power supplied to the heater layer 34 and/or to the cooling system 14. In one embodiment, the processor 74 provides a control signal through a digital I/O device 77 to a pulse-width modulator (PWM) control 80, which provides a signal that controls the operation of the current drivers 78. Alternatively, a low-pass filter (not shown) at the output of the PWM provides for continuous operation of the current drivers 78.

In another embodiment, temperature sensors may be located at the cooling system 14 and appropriately connected to the A/D system(s) and processor to provide closed-loop control of the cooling system as well.

In yet another embodiment, a detector cooling system 82 is located in thermally conductive relation to one or more of the detectors 28. The detector cooling system 82 may comprise any of the devices disclosed above as comprising the cooling system 14, and preferably comprises a Peltier-type thermoelectric device. The temperature control subsystem may also include temperature sensors, such as RTDs and/or thermistors, located in or adjacent to the detector cooling system 82, and electrical connections between these sensors and the asynchronous A/D system 72. The temperature sensors of the detector cooling system 82 provide detector temperature signals to the processor 74. In one embodiment, the detector cooling system 82 operates independently of the window temperature control system, and the detector cooling system temperature signals are sampled using the asynchronous A/D system 72. In accordance with the temperature control algorithm, the processor 74 determines appropriate control inputs for the detector cooling system 82, based on the information contained in the detector temperature signal. The processor 74 outputs digital control signals to the D/A system 76 which in turn provides analog control signals to the current drivers 78. In response to the control signals, the current drivers 78 regulate the power supplied to the detector cooling system 14. In one embodiment, the processor 74 also provides a control signal through the digital I/O device 77 and the PWM control 80, to control the operation of the detector cooling system 82 by the current drivers 78. Alternatively, a low-pass filter (not shown) at the output of the PWM provides for continuous operation of the current drivers 78.

In the data acquisition subsystem, the detectors 28 respond to the infrared energy E incident thereon by passing one or more analog detector signals to a preamp 84. The preamp 84 amplifies the detector signals and passes them to the synchronous A/D system 70, which converts the detector signals to digital form and passes them to the processor 74. The processor 74 determines the concentrations of the analyte(s) of interest, based on the detector signals and a concentration-analysis algorithm and/or phase/concentration regression model stored in a memory module 88. The concentration-analysis algorithm and/or phase/concentration regression model may be developed according to any of the analysis methodologies discussed herein. The processor may communicate the concentration results and/or other information to a display controller 86, which operates a display (not shown), such as an LCD display, to present the information to the user.

A watchdog timer 94 may be employed to ensure that the processor 74 is operating correctly. If the watchdog timer 94 does not receive a signal from the processor 74 within a specified time, the watchdog timer 94 resets the processor 74. The control system may also include a JTAG interface 96 to enable testing of the noninvasive system 10.

In one embodiment, the synchronous A/D system 70 comprises a 20-bit, 14 channel system, and the asynchronous A/D system 72 comprises a 16-bit, 16 channel system. The preamp may comprise a 12-channel preamp corresponding to an array of 12 detectors 28.

The control system may also include a serial port 90 or other conventional data port to permit connection to a personal computer 92. The personal computer can be employed to update the algorithm(s) and/or phase/concentration regression model(s) stored in the memory module 88, or to download a compilation of analyte-concentration data from the noninvasive system. A real-time clock or other timing device may be accessible by the processor 74 to make any time-dependent calculations which may be desirable to a user.

2. Analysis Methodology

The detector(s) 28 of the noninvasive system 10 are used to detect the infrared energy emitted by the material sample S in various desired wavelengths. At each measured wavelength, the material sample S emits infrared energy at an intensity which varies over time. The time-varying intensities arise largely in response to the use of the window assembly 12 (including its heater layer 34) and the cooling system 14 to induce a thermal gradient in the material sample S. As used herein, “thermal gradient” is a broad term and is used in its ordinary sense and refers, without limitation, to a difference in temperature and/or thermal energy between different locations, such as different depths, of a material sample, which can be induced by any suitable method of increasing or decreasing the temperature and/or thermal energy in one or more locations of the-sample. As will be discussed in detail below, the concentration of an analyte of interest (such as glucose) in the material sample S can be determined with a device such as the noninvasive system 10, by comparing the time-varying intensity profiles of the various measured wavelengths.

Analysis methodologies are discussed herein within the context of detecting the concentration of glucose within a material sample, such as a tissue sample, which includes a large proportion of water. However, it will evident that these methodologies are not limited to this context and may be applied to the detection of a wide variety of analytes within a wide variety of sample types. It should also be understood that other suitable analysis methodologies and suitable variations of the disclosed methodologies may be employed in operating an analyte detection system, such as the noninvasive system 10.

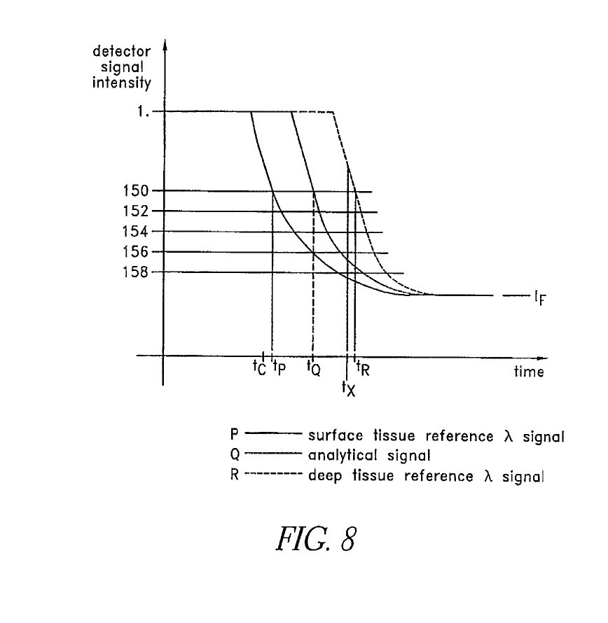

As shown in FIG. 8, a first reference signal P may be measured at a first reference wavelength. The first reference signal P is measured at a wavelength where water strongly absorbs (e.g., 2.9 [symbol] m or 6.1 [symbol] m). Because water strongly absorbs radiation at these wavelengths, the detector signal intensity is reduced at those wavelengths. Moreover, at these wavelengths water absorbs the photon emissions emanating from deep inside the sample. The net effect is that a signal emitted at these wavelengths from deep inside the sample is not easily detected. The first reference signal P is thus a good indicator of thermal-gradient effects near the sample surface and may be known as a surface reference signal. This signal may be calibrated and normalized, in the absence of heating or cooling applied to the sample, to a baseline value of 1. For greater accuracy, more than one first reference wavelength may be measured. For example, both 2.9 [symbol] m and 6.1 [symbol] m may be chosen as first reference wavelengths.

As further shown in FIG. 8, a second reference signal R may also be measured. The second signal R may be measured at a wavelength where water has very low absorbance (e.g., 3.6 [symbol] m or 4.2 [symbol] m). This second reference signal R thus provides the analyst with information concerning the deeper regions of the sample, whereas the first signal P provides information concerning the sample surface. This signal may also be calibrated and normalized, in the absence of heating or cooling applied to the sample, to a baseline value of 1. As with the first (surface) reference signal P, greater accuracy may be obtained by using more than one second (deep) reference signal R.

In order to determine analyte concentration, a third (analytical) signal Q is also measured. This signal is measured at an IR absorbance peak of the selected analyte. The IR absorbance peaks for glucose are in the range of about 6.5 [symbol] m to 11.0 [symbol] m. This detector signal may also be calibrated and normalized, in the absence of heating or cooling applied to the material sample S, to a baseline value of 1. As with the reference signals P, R, the analytical signal Q may be measured at more than one absorbance peak.

Optionally, or additionally, reference signals may be measured at wavelengths that bracket the analyte absorbance peak. These signals may be advantageously monitored at reference wavelengths which do not overlap the analyte absorbance peaks. Further, it is advantageous to measure reference wavelengths at absorbance peaks which do not overlap the absorbance peaks of other possible constituents contained in the sample.

a. Basic Thermal Gradient

As further shown in FIG. 8, the signal intensities P, Q, R are shown initially at the normalized baseline signal intensity of 1. This of course reflects the baseline radiative behavior of a test sample in the absence of applied heating or cooling. At a time t_{C}, the surface of the sample is subjected to a temperature event which induces a thermal gradient in the sample. The gradient can be induced by heating or cooling the sample surface. The example shown in FIG. 8 uses cooling, for example, using a 10° C. cooling event. In response to the cooling event, the intensities of the detector signals P, Q, R decrease over time.

Since the cooling of the sample is neither uniform nor instantaneous, the surface cools before the deeper regions of the sample cool. As each of the signals P, Q, R drop in intensity, a pattern emerges. Signal intensity declines as expected, but as the signals P, Q, R reach a given amplitude value (or series of amplitude values: 150, 152, 154, 156, 158), certain temporal effects are noted. After the cooling event is induced at t_{C}, the first (surface) reference signal P declines in amplitude most rapidly, reaching a checkpoint 150 first, at time t_{P}. This is due to the fact that the first reference signal P mirrors the sample's radiative characteristics near the surface of the sample. Since the sample surface cools before the underlying regions, the surface (first) reference signal P drops in intensity first.

Simultaneously, the second reference signal R is monitored. Since the second reference signal R corresponds to the radiation characteristics of deeper regions of the sample, which do not cool as rapidly as the surface (due to the time needed for the surface cooling to propagate into the deeper regions of the sample), the intensity of signal R does not decline until slightly later. Consequently, the signal R does not reach the magnitude 150 until some later time t_{R}. In other words, there exists a time delay between the time t_{P} at which the amplitude of the first reference signal P reaches the checkpoint 150 and the time t_{R} at which the second reference signal R reaches the same checkpoint 150. This time delay can be expressed as a phase difference Ö(ë). Additionally, a phase difference may be measured between the analytical signal Q and either or both reference signals P, R.

As the concentration of analyte increases, the amount of absorbance at the analytical wavelength increases. This reduces the intensity of the analytical signal Q in a concentration-dependent way. Consequently, the analytical signal Q reaches intensity 150 at some intermediate time t_{Q}. The higher the concentration of analyte, the more the analytical signal Q shifts to the left in FIG. 8. As a result, with increasing analyte concentration, the phase difference Ö(ë) decreases relative to the first (surface) reference signal P and increases relative to the second (deep tissue) reference signal R. The phase difference(s) Ö(ë) are directly related to analyte concentration and can be used to make accurate determinations of analyte concentration.

The phase difference Ö(ë) between the first (surface) reference signal P and the analytical signal Q is represented by the equation:

\ddot{O}(\ddot{e})=|t_{P}−t_{Q}|

The magnitude of this phase difference decreases with increasing analyte concentration.

The phase difference Ö(ë) between the second (deep tissue) reference signal R and the analytical signal Q signal is represented by the equation:

\ddot{O}(\ddot{e})=|t_{Q}−t_{R}|

The magnitude of this phase difference increases with increasing analyte concentration.

Accuracy may be enhanced by choosing several checkpoints, for example, 150, 152, 154, 156, and 158 and averaging the phase differences observed at each checkpoint. The accuracy of this method may be further enhanced by integrating the phase difference(s) continuously over the entire test period. Because in this example only a single temperature event (here, a cooling event) has been induced, the sample reaches a new lower equilibrium temperature and the signals stabilize at a new constant level I_{F}. Of course, the method works equally well with thermal gradients induced by heating or by the application or introduction of other forms of energy, such as but not limited to light, radiation, chemically induced heat, friction and vibration.

This methodology is not limited to the determination of phase difference. At any given time (for example, at a time t_{X}) the amplitude of the analytical signal Q may be compared to the amplitude of either or both of the reference signals P, R. The difference in amplitude may be observed and processed to determine analyte concentration.

This method, the variants disclosed herein, and the apparatus disclosed as suitable for application of the method(s), are not limited to the detection of in-vivo glucose concentration. The method and disclosed variants and apparatus may be used on human, animal, or even plant subjects, or on organic or inorganic compositions in a non-medical setting. The method may be used to take measurements of in-vivo or in-vitro samples of virtually any kind. The method is useful for measuring the concentration of a wide range of additional chemical analytes, including but not limited to, glucose, ethanol, insulin, water, carbon dioxide, blood oxygen, cholesterol, bilirubin, ketones, fatty acids, lipoproteins, albumin, urea, creatinine, white blood cells, red blood cells, hemoglobin, oxygenated hemoglobin, carboxyhemoglobin, organic molecules, inorganic molecules, pharmaceuticals, cytochrome, various proteins and chromophores, microcalcifications, hormones, as well as other chemical compounds. To detect a given analyte, one needs only to select appropriate analytical and reference wavelengths.

The method is adaptable and may be used to determine chemical concentrations in samples of body fluids (e.g., blood, urine or saliva) once they have been extracted from a patient. In fact, the method may be used for the measurement of in-vitro samples of virtually any kind.

b. Modulated Thermal Gradient

In a variation of the methodology described above, a periodically modulated thermal gradient can be employed to make accurate determinations of analyte concentration.

As previously shown in FIG. 8, once a thermal gradient is induced in the sample, the reference and analytical signals P, Q, R fall out of phase with respect to each other. This phase difference Ö(ë) is present whether the thermal gradient is induced through heating or cooling. By alternatively subjecting the test sample to cyclic pattern of heating, cooling, or alternately heating and cooling, an oscillating thermal gradient may be induced in a sample for an extended period of time.

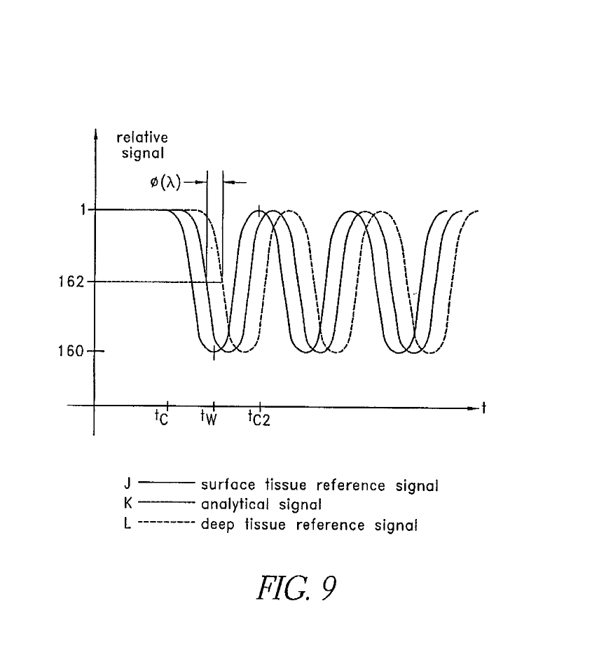

An oscillating thermal gradient is illustrated using a sinusoidally modulated gradient. FIG. 9 depicts detector signals emanating from a test sample. As with the methodology shown in FIG. 8, one or more reference signals J, L are measured. One or more analytical signals K are also monitored. These signals may be calibrated and normalized, in the absence of heating or cooling applied to the sample, to a baseline value of 1. FIG. 9 shows the signals after normalization. At some time t_{C}, a temperature event (e.g., cooling) is induced at the sample surface. This causes a decline in the detector signal. As shown in FIG. 8, the signals (P, Q, R) decline until the thermal gradient disappears and a new equilibrium detector signal I_{F} is reached. In the method shown in FIG. 9, as the gradient begins to disappear at a signal intensity 160, a heating event, at a time t_{W}, is induced in the sample surface. As a result the detector output signals J, K, L will rise as the sample temperature rises. At some later time t_{C2}, another cooling event is induced, causing the temperature and detector signals to decline. This cycle of cooling and heating may be repeated over a time interval of arbitrary length. Moreover, if the cooling and heating events are timed properly, a periodically modulated thermal gradient may be induced in the test sample.

As previously explained in the discussions relating to FIG. 8, the phase difference Ö(ë) may be measured and used to determine analyte concentration. FIG. 9 shows that the first (surface) reference signal J declines and rises in intensity first. The second (deep tissue) reference signal L declines and rises in a time-delayed manner relative to the first reference signal J. The analytical signal K exhibits a time/phase delay dependent on the analyte concentration. With increasing concentration, the analytical signal K shifts to the left in FIG. 9. As with FIG. 8, the phase difference Ö(ë) may be measured. For example, a phase difference Ö(ë) between the second reference signal L and the analytical signal K, may be measured at a set amplitude 162 as shown in FIG. 9. Again, the magnitude of the phase signal reflects the analyte concentration of the sample.

The phase-difference information compiled by any of the methodologies disclosed herein can correlated by the control system 30 (see FIG. 1) with previously determined phase-difference information to determine the analyte concentration in the sample. This correlation could involve comparison of the phase-difference information received from analysis of the sample, with a data set containing the phase-difference profiles observed from analysis of wide variety of standards of known analyte concentration. In one embodiment, a phase/concentration curve or regression model is established by applying regression techniques to a set of phase-difference data observed in standards of known analyte concentration. This curve is used to estimate the analyte concentration in a sample based on the phase-difference information received from the sample.

Advantageously, the phase difference Ö(ë) may be measured continuously throughout the test period. The phase-difference measurements may be integrated over the entire test period for an extremely accurate measure of phase difference Ö(ë). Accuracy may also be improved by using more than one reference signal and/or more than one analytical signal.

As an alternative or as a supplement to measuring phase difference(s), differences in amplitude between the analytical and reference signal(s) may be measured and employed to determine analyte concentration. Additional details relating to this technique and not necessary to repeat here may be found in the Assignee's U.S. patent application Ser. No. 09/538,164, incorporated by reference below.

Additionally, these methods may be advantageously employed to simultaneously measure the concentration of one or more analytes. By choosing reference and analyte wavelengths that do not overlap, phase differences can be simultaneously measured and processed to determine analyte concentrations. Although FIG. 9 illustrates the method used in conjunction with a sinusoidally modulated thermal gradient, the principle applies to thermal gradients conforming to any periodic function. In more complex cases, analysis using signal processing with Fourier transforms or other techniques allows accurate determinations of phase difference Ö(ë) and analyte concentration.

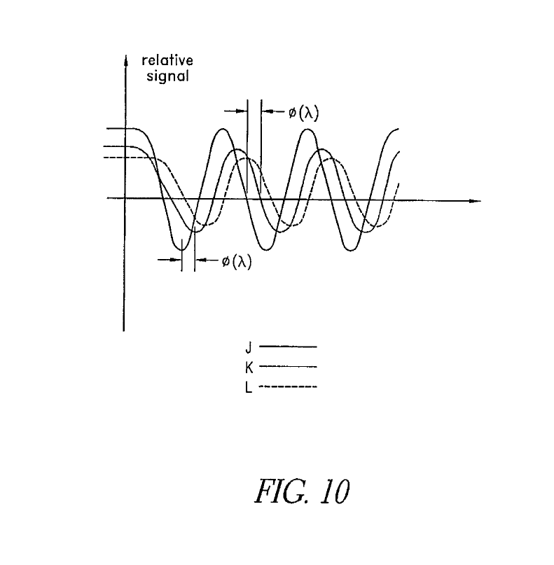

As shown in FIG. 10, the magnitude of the phase differences may be determined by measuring the time intervals between the amplitude peaks (or troughs) of the reference signals J, L and the analytical signal K. Alternatively, the time intervals between the “zero crossings” (the point at which the signal amplitude changes from positive to negative, or negative to positive) may be used to determine the phase difference between the analytical signal K and the reference signals J, L. This information is subsequently processed and a determination of analyte concentration may then be made. This particular method has the advantage of not requiring normalized signals.

As a further alternative, two or more driving frequencies may be employed to determine analyte concentrations at selected depths within the sample. A slow (e.g., 1 Hz) driving frequency creates a thermal gradient which penetrates deeper into the sample than the gradient created by a fast (e.g., 3 Hz) driving frequency. This is because the individual heating and/or cooling events are longer in duration where the driving frequency is lower. Thus, the use of a slow driving frequency provides analyte-concentration information from a deeper “slice” of the sample than does the use of a fast driving frequency.

It has been found that when analyzing a sample of human skin, a temperature event of 10° C. creates a thermal gradient which penetrates to a depth of about 150 μm, after about 500 ms of exposure. Consequently, a cooling/heating cycle or driving frequency of 1 Hz provides information to a depth of about 150 μm. It has also been determined that exposure to a temperature event of 10° C. for about 167 ms creates a thermal gradient that penetrates to a depth of about 50 μm. Therefore, a cooling/heating cycle of 3 Hz provides information to a depth of about 50 μm. By subtracting the detector signal information measured at a 3 Hz driving frequency from the detector signal information measured at a 1 Hz driving frequency, one can determine the analyte concentration(s) in the region of skin between 50 and 150 μm. Of course, a similar approach can be used to determine analyte concentrations at any desired depth range within any suitable type of sample.

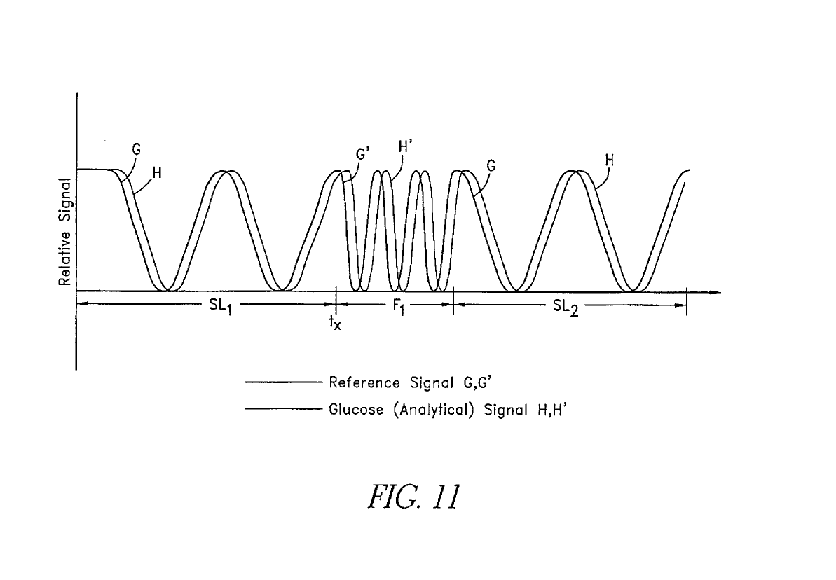

As shown in FIG. 11, alternating deep and shallow thermal gradients may be induced by alternating slow and fast driving frequencies. As with the methods described above, this variation also involves the detection and measurement of phase differences Ö(ë) between reference signals G, G′ and analytical signals H, H′. Phase differences are measured at both fast (e.g., 3 Hz) and slow (e.g., 1 Hz) driving frequencies. The slow driving frequency may continue for an arbitrarily chosen number of cycles (in region SL₁), for example, two full cycles. Then the fast driving frequency is employed for a selected duration, in region F₁. The phase difference data is compiled in the same manner as disclosed above. In addition, the fast frequency (shallow sample) phase difference data may be subtracted from the slow frequency (deep sample) data to provide an accurate determination of analyte concentration in the region of the sample between the gradient penetration depth associated with the fast driving frequency and that associated with the slow driving frequency.

The driving frequencies (e.g., 1 Hz and 3 Hz) can be multiplexed as shown in FIG. 12. The fast (3 Hz) and slow (1 Hz) driving frequencies can be superimposed rather than sequentially implemented. During analysis, the data can be separated by frequency (using Fourier transform or other techniques) and independent measurements of phase delay at each of the driving frequencies may be calculated. Once resolved, the two sets of phase delay data are processed to determine absorbance and analyte concentration.

Additional details not necessary to repeat here may be found in U.S. Pat. No. 6,198,949, titled SOLID-STATE NON-INVASIVE INFRARED ABSORPTION SPECTROMETER FOR THE GENERATION AND CAPTURE OF THERMAL GRADIENT SPECTRA FROM LIVING TISSUE, issued Mar. 6, 2001; U.S. Pat. No. 6,161,028, titled METHOD FOR DETERMINING ANALYTE CONCENTRATION USING PERIODIC TEMPERATURE MODULATION AND PHASE DETECTION, issued Dec. 12, 2000; U.S. Pat. No. 5,877,500, titled MULTICHANNEL INFRARED DETECTOR WITH OPTICAL CONCENTRATORS FOR EACH CHANNEL, issued on Mar. 2, 1999; U.S. patent application Ser. No. 09/538,164, filed Mar. 30, 2000 and titled METHOD AND APPARATUS FOR DETERMINING ANALYTE CONCENTRATION USING PHASE AND MAGNITUDE DETECTION OF A RADIATION TRANSFER FUNCTION; U.S. Provisional Patent Application No. 60/336,404, filed Oct. 29, 2001, titled WINDOW ASSEMBLY; U.S. Provisional Patent Application No. 60/340,435, filed Dec. 12, 2001, titled CONTROL SYSTEM FOR BLOOD CONSTITUENT MONITOR; U.S. Provisional Patent Application No. 60/340,654, filed Dec. 12, 2001, titled SYSTEM AND METHOD FOR CONDUCTING AND DETECTING INFRARED RADIATION; U.S. Provisional Patent Application No. 60/336,294, filed Oct. 29, 2001, titled METHOD AND DEVICE FOR INCREASING ACCURACY OF BLOOD CONSTITUENT MEASUREMENT; and U.S. Provisional Patent Application No. 60/339,116, filed Nov. 7, 2001, titled METHOD AND APPARATUS FOR IMPROVING CLINICALLY SIGNIFICANT ACCURACY OF ANALYTE MEASUREMENTS. The entire disclosure of all of the above-mentioned patents, patent applications and publications is hereby incorporated by reference herein and made a part of this specification.

### B. Whole-Blood Detection System

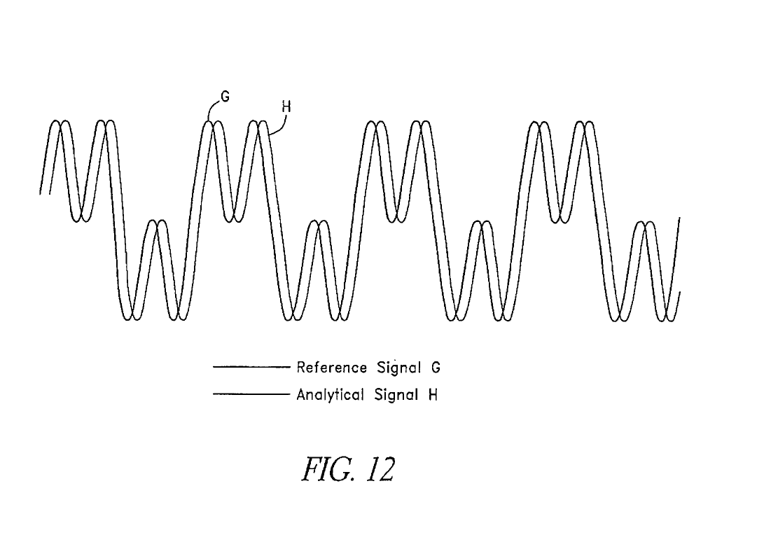

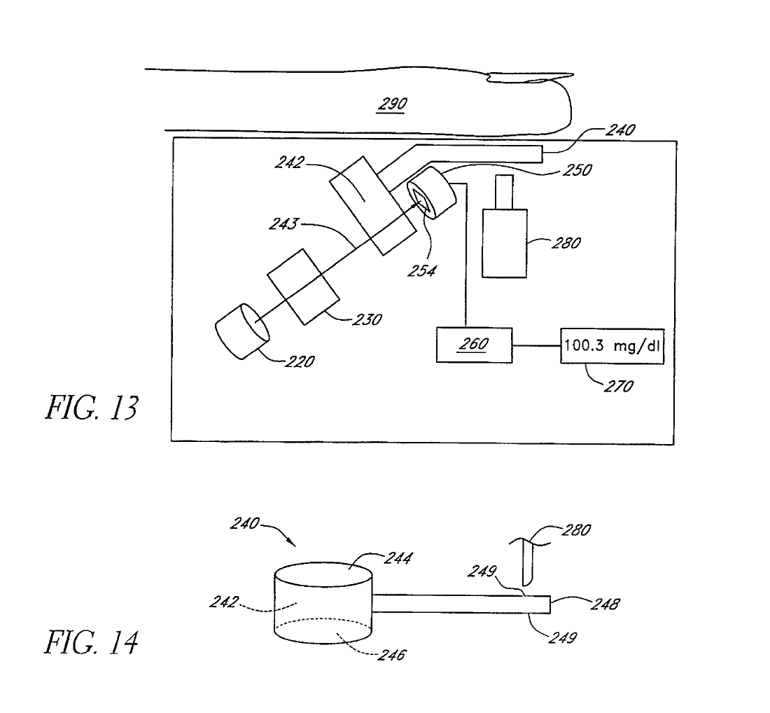

FIG. 13 is a schematic view of a reagentless whole-blood analyte detection system 200 (hereinafter “whole-blood system”) in a preferred configuration. The whole-blood system 200 may comprise a radiation source 220, a filter 230, a cuvette 240 that includes a sample cell 242, and a radiation detector 250. The whole-blood system 200 preferably also comprises a signal processor 260 and a display 270. Although a cuvette 240 is shown here, other sample elements, as described below, could also be used in the system 200. The whole-blood system 200 can also comprise a sample extractor 280, which can be used to access bodily fluid from an appendage, such as the finger 290, forearm, or any other suitable location.

As used herein, the terms “whole-blood analyte detection system” and “whole-blood system” are broad, synonymous terms and are used in their ordinary sense and refer, without limitation, to analyte detection devices which can determine the concentration of an analyte in a material sample by passing electromagnetic radiation through the sample and detecting the absorbance of the radiation by the sample. As used herein, the term “whole-blood” is a broad term and is used in its ordinary sense and refers, without limitation, to blood that has been withdrawn from a patient but that has not been otherwise processed, e.g., it has not been hemolysed, lyophilized, centrifuged, or separated in any other manner, after being removed from the patient. Whole-blood may contain amounts of other fluids, such as interstitial fluid or intracellular fluid, which may enter the sample during the withdrawal process or are naturally present in the blood. It should be understood, however, that the whole-blood system 200 disclosed herein is not limited to analysis of whole-blood, as the whole-blood system 10 may be employed to analyze other substances, such as saliva, urine, sweat, interstitial fluid, intracellular fluid, hemolysed, lyophilized, or centrifuged blood or any other organic or inorganic materials.

The whole-blood system 200 may comprise a near-patient testing system. As used herein, “near-patient testing system” is a broad term and is used in its ordinary sense, and includes, without limitation, test systems that are configured to be used where the patient is rather than exclusively in a laboratory, e.g., systems that can be used at a patient's home, in a clinic, in a hospital, or even in a mobile environment. Users of near-patient testing systems can include patients, family members of patients, clinicians, nurses, or doctors. A “near-patient testing system” could also include a “point-of-care” system.

The whole-blood system 200 may in one embodiment be configured to be operated easily by the patient or user. As such, the system 200 is preferably a portable device. As used herein, “portable” is a broad term and is used in its ordinary sense and means, without limitation, that the system 200 can be easily transported by the patient and used where convenient. For example, the system 200 is advantageously small. In one preferred embodiment, the system 200 is small enough to fit into a purse or backpack. In another embodiment, the system 200 is small enough to fit into a pants pocket. In still another embodiment, the system 200 is small enough to be held in the palm of a hand of the user.

Some of the embodiments described herein employ a sample element to hold a material sample, such as a sample of biological fluid. As used herein, “sample element” is a broad term and is used in its ordinary sense and includes, without limitation, structures that have a sample cell and at least one sample cell wall, but more generally includes any of a number of structures that can hold, support or contain a material sample and that allow electromagnetic radiation to pass through a sample held, supported or contained thereby; e.g., a cuvette, test strip, etc. As used herein, the term “disposable” when applied to a component, such as a sample element, is a broad term and is used in its ordinary sense and means, without limitation, that the component in question is used a finite number of times and then discarded. Some disposable components are used only once and then discarded. Other disposable components are used more than once and then discarded.

The radiation source 220 of the whole-blood system 200 emits electromagnetic radiation in any of a number of spectral ranges, e.g., within infrared wavelengths; in the mid-infrared wavelengths; above about 0.8 [symbol] m; between about 5.0 [symbol] m and about 20.0 [symbol] m; and/or between about 5.25 [symbol] m and about 12.0 [symbol] m. However, in other embodiments the whole-blood system 200 may employ a radiation source 220 which emits in wavelengths found anywhere from the visible spectrum through the microwave spectrum, for example anywhere from about 0.4 [symbol] m to greater than about 100 [symbol] m. In still further embodiments the radiation source emits electromagnetic radiation in wavelengths between about 3.5 [symbol] m and about 14 [symbol] m, or between about 0.8 [symbol] m and about 2.5 [symbol] m, or between about 2.5 [symbol] m and about 20 [symbol] m, or between about 20 [symbol] m and about 100 [symbol] m, or between about 6.85 [symbol] m and about 10.10 [symbol] m.

The radiation emitted from the source 220 is in one embodiment modulated at a frequency between about one-half hertz and about one hundred hertz, in another embodiment between about 2.5 hertz and about 7.5 hertz, in still another embodiment at about 50 hertz, and in yet another embodiment at about 5 hertz. With a modulated radiation source, ambient light sources, such as a flickering fluorescent lamp, can be more easily identified and rejected when analyzing the radiation incident on the detector 250. One source that is suitable for this application is produced by ION OPTICS, INC. and sold under the part number NL5LNC.

The filter 230 permits electromagnetic radiation of selected wavelengths to pass through and impinge upon the cuvette/sample element 240. Preferably, the filter 230 permits radiation at least at about the following wavelengths to pass through to the cuvette/sample element: 3.9, 4.0 [symbol] m, 4.05 [symbol] m, 4.2 [symbol] m, 4.75, 4.95 [symbol] m, 5.25 [symbol] m, 6.12 [symbol] m, 7.4 [symbol] m, 8.0 [symbol] m, 8.45 [symbol] m, 9.25 [symbol] m, 9.5 [symbol] m, 9.65 [symbol] m, 10.4 [symbol] m, 12.2 [symbol] m. In another embodiment, the filter 230 permits radiation at least at about the following wavelengths to pass through to the cuvette/sample element: 5.25 [symbol] m, 6.12 [symbol] m, 6.8 [symbol] m, 8.03 [symbol] m, 8.45 [symbol] m, 9.25 [symbol] m, 9.65 [symbol] m, 10.4 [symbol] m, 12 [symbol] m. In still another embodiment, the filter 230 permits radiation at least at about the following wavelengths to pass through to the cuvette/sample element: 6.85 [symbol] m, 6.97 [symbol] m, 7.39 [symbol] m, 8.23 [symbol] m, 8.62 [symbol] m, 9.02 [symbol] m, 9.22 [symbol] m, 9.43 [symbol] m, 9.62 [symbol] m, and 10.10 [symbol] m. The sets of wavelengths recited above correspond to specific embodiments within the scope of this disclosure. Furthermore, other subsets of the foregoing sets or other combinations of wavelengths can be selected. Finally, other sets of wavelengths can be selected within the scope of this disclosure based on cost of production, development time, availability, and other factors relating to cost, manufacturability, and time to market of the filters used to generate the selected wavelengths, and/or to reduce the total number of filters needed.

In one embodiment, the filter 230 is capable of cycling its passband among a variety of narrow spectral bands or a variety of selected wavelengths. The filter 230 may thus comprise a solid-state tunable infrared filter, such as that available from ION OPTICS INC. The filter 230 could also be implemented as a filter wheel with a plurality of fixed-passband filters mounted on the wheel, generally perpendicular to the direction of the radiation emitted by the source 220. Rotation of the filter wheel alternately presents filters that pass radiation at wavelengths that vary in accordance with the filters as they pass through the field of view of the detector 250.

The detector 250 preferably comprises a 3 mm long by 3 mm wide pyroelectric detector. Suitable examples are produced by DIAS Angewandte Sensorik GmbH of Dresden, Germany, or by BAE Systems (such as its TGS model detector). The detector 250 could alternatively comprise a thermopile, a bolometer, a silicon microbolometer, a lead-salt focal plane array, or a mercury-cadmium-telluride (MCT) detector. Whichever structure is used as the detector 250, it is desirably configured to respond to the radiation incident upon its active surface 254 to produce electrical signals that correspond to the incident radiation.

In one embodiment, the sample element comprises a cuvette 240 which in turn comprises a sample cell 242 configured to hold a sample of tissue and/or fluid (such as whole-blood, blood components, interstitial fluid, intercellular fluid, saliva, urine, sweat and/or other organic or inorganic materials) from a patient within its sample cell. The cuvette 240 is installed in the whole-blood system 200 with the sample cell 242 located at least partially in the optical path 243 between the radiation source 220 and the detector 250. Thus, when radiation is emitted from the source 220 through the filter 230 and the sample cell 242 of the cuvette 240, the detector 250 detects the radiation signal strength at the wavelength(s) of interest. Based on this signal strength, the signal processor 260 determines the degree to which the sample in the cell 242 absorbs radiation at the detected wavelength(s). The concentration of the analyte of interest is then determined from the absorption data via any suitable spectroscopic technique.

As shown in FIG. 13, the whole-blood system 200 can also comprise a sample extractor 280. As used herein, the term “sample extractor” is a broad term and is used in its ordinary sense and refers, without limitation, to any device which is suitable for drawing a sample material, such as whole-blood, other bodily fluids, or any other sample material, through the skin of a patient. In various embodiments, the sample extractor may comprise a lance, laser lance, iontophoretic sampler, gas-jet, fluid-jet or particle-jet perforator, ultrasonic enhancer (used with or without a chemical enhancer), or any other suitable device.

As shown in FIG. 13, the sample extractor 280 could form an opening in an appendage, such as the finger 290, to make whole-blood available to the cuvette 240. It should be understood that other appendages could be used to draw the sample, including but not limited to the forearm. With some embodiments of the sample extractor 280, the user forms a tiny hole or slice through the skin, through which flows a sample of bodily fluid such as whole-blood. Where the sample extractor 280 comprises a lance (see FIG. 14), the sample extractor 280 may comprise a sharp cutting implement made of metal or other rigid materials. One suitable laser lance is the Lasette Plus® produced by Cell Robotics International, Inc. of Albuquerque, N. Mex. If a laser lance, iontophoretic sampler, gas-jet or fluidjet perforator is used as the sample extractor 280, it could be incorporated into the whole-blood system 200 (see FIG. 13), or it could be a separate device.

Additional information on laser lances can be found in U.S. Pat. No. 5,908,416, issued Jun. 1, 1999, titled LASER DERMAL PERFORATOR; the entirety of this patent is hereby incorporated by reference herein and made a part of this specification. One suitable gas-jet, fluid-jet or particle-jet perforator is disclosed in U.S. Pat. No. 6,207,400, issued Mar. 27, 2001, titled NON- OR MINIMALLY INVASIVE MONITORING METHODS USING PARTICLE DELIVERY METHODS; the entirety of this patent is hereby incorporated by reference herein and made a part of this specification. One suitable iontophoretic sampler is disclosed in U.S. Pat. No. 6,298,254, issued Oct. 2, 2001, titled DEVICE FOR SAMPLING SUBSTANCES USING ALTERNATING POLARITY OF IONTOPHORETIC CURRENT; the entirety of this patent is hereby incorporated by reference herein and made a part of this specification. One suitable ultrasonic enhancer, and chemical enhancers suitable for use therewith, are disclosed in U.S. Pat. No. 5,458,140, titled ENHANCEMENT OF TRANSDERMAL MONITORING APPLICATIONS WITH ULTRASOUND AND CHEMICAL ENHANCERS, issued Oct. 17, 1995, the entire disclosure of which is hereby incorporated by reference and made a part of this specification.

FIG. 14 shows one embodiment of a sample element, in the form of a cuvette 240, in greater detail. The cuvette 240 further comprises a sample supply passage 248, a pierceable portion 249, a first window 244, and a second window 246, with the sample cell 242 extending between the windows 244, 246. In one embodiment, the cuvette 240 does not have a second window 246. The first window 244 (or second window 246) is one form of a sample cell wall; in other embodiments of the sample elements and cuvettes disclosed herein, any sample cell wall may be used that at least partially contains, holds or supports a material sample, such as a biological fluid sample, and which is transmissive of at least some bands of electromagnetic radiation, and which may but need not be transmissive of electromagnetic radiation in the visible range. The pierceable portion 249 is an area of the sample supply passage 248 that can be pierced by suitable embodiments of the sample extractor 280. Suitable embodiments of the sample extractor 280 can pierce the portion 249 and the appendage 290 to create a wound in the appendage 290 and to provide an inlet for the blood or other fluid from the wound to enter the cuvette 240. (The sample extractor 280 is shown on the opposite side of the sample element in FIG. 14, as compared to FIG. 13, as it may pierce the portion 249 from either side.)

The windows 244, 246 are preferably optically transmissive in the range of electromagnetic radiation that is emitted by the source 220, or that is permitted to pass through the filter 230. In one embodiment, the material that makes up the windows 244, 246 is completely transmissive, i.e., it does not absorb any of the electromagnetic radiation from the source 220 and filter 230 that is incident upon it. In another embodiment, the material of the windows 244, 246 has some absorption in the electromagnetic range of interest, but its absorption is negligible. In yet another embodiment, the absorption of the material of the windows 244, 246 is not negligible, but it is known and stable for a relatively long period of time. In another embodiment, the absorption of the windows 244, 246 is stable for only a relatively short period of time, but the whole-blood system 200 is configured to observe the absorption of the material and eliminate it from the analyte measurement before the material properties can change measurably.

The windows 244, 246 are made of polypropylene in one embodiment. In another embodiment, the windows 244, 246 are made of polyethylene. Polyethylene and polypropylene are materials having particularly advantageous properties for handling and manufacturing, as is known in the art. Also, polypropylene can be arranged in a number of structures, e.g., isotactic, atactic and syndiotactic, which may enhance the flow characteristics of the sample in the sample element. Preferably the windows 244, 246 are made of durable and easily manufactureable materials, such as the above-mentioned polypropylene or polyethylene, or silicon or any other suitable material. The windows 244, 246 can be made of any suitable polymer, which can be isotactic, atactic or syndiotactic in structure.

The distance between the windows 244, 246 comprises an optical pathlength and can be between about 1 [symbol] m and about 100 [symbol] m. In one embodiment, the optical pathlength is between about 10 [symbol] m and about 40 [symbol] m, or between about 25 [symbol] m and about 60 [symbol] m, or between about 30 [symbol] m and about 50 [symbol] m. In still another embodiment, the optical pathlength is about 25 [symbol] m. The transverse size of each of the windows 244, 246 is preferably about equal to the size of the detector 250. In one embodiment, the windows are round with a diameter of about 3 mm. In this embodiment, where the optical pathlength is about 25 [symbol] m the volume of the sample cell 242 is about 0.177 i L. In one embodiment, the length of the sample supply passage 248 is about 6 mm, the height of the sample supply passage 248 is about 1 mm, and the thickness of the sample supply passage 248 is about equal to the thickness of the sample cell, e.g., 25 [symbol] m. The volume of the sample supply passage is about 0.150 i L. Thus, the total volume of the cuvette 240 in one embodiment is about 0.327 i L. Of course, the volume of the cuvette 240/sample cell 242/etc. can vary, depending on many variables, such as the size and sensitivity of the detectors 250, the intensity of the radiation emitted by the source 220, the expected flow properties of the sample, and whether flow enhancers (discussed below) are incorporated into the cuvette 240. The transport of fluid to the sample cell 242 is achieved preferably through capillary action, but may also be achieved through wicking, or a combination of wicking and capillary action.

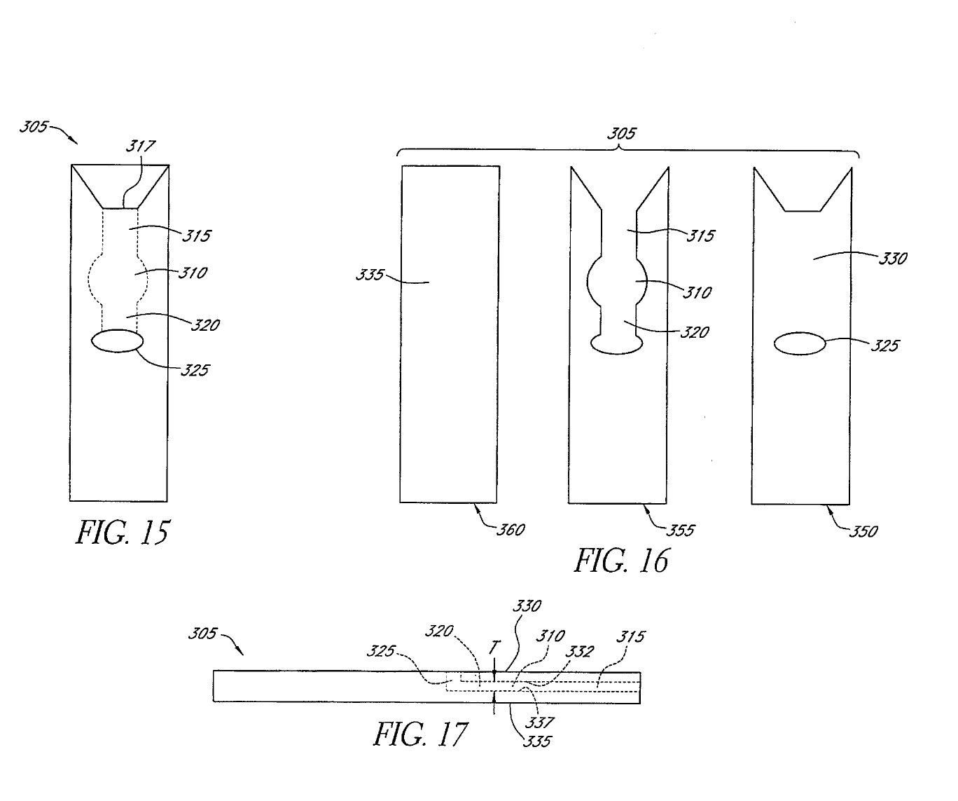

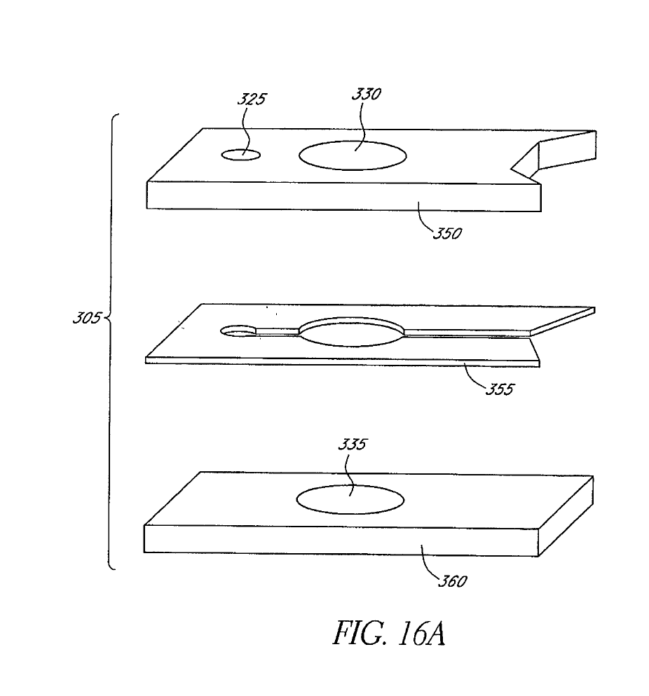

FIGS. 15-17 depict another embodiment of a cuvette 305 that could be used in connection with the whole-blood system 200. The cuvette 305 comprises a sample cell 310, a sample supply passage 315, an air vent passage 320, and a vent 325. As best seen in FIGS. 16,16A and 17, the cuvette also comprises a first sample cell window 330 having an inner side 332, and a second sample cell window 335 having an inner side 337. As discussed above, the window(s) 330/335 in some embodiments also comprise sample cell wall(s). The cuvette 305 also comprises an opening 317 at the end of the sample supply passage 315 opposite the sample cell 310. The cuvette 305 is preferably about ¼ - ⅛ inch wide and about ¾ inch long; however, other dimensions are possible while still achieving the advantages of the cuvette 305.

The sample cell 310 is defined between the inner side 332 of the first sample cell window 330 and the inner side 337 of the second sample cell window 335. The perpendicular distance T between the two inner sides 332, 337 comprises an optical pathlength that can be between about 1 [symbol] m and about 1.22 mm. The optical pathlength can alternatively be between about 1 [symbol] m and about 100 [symbol] m. The optical pathlength could still alternatively be about 80 [symbol] m, but is preferably between about 10 [symbol] m and about 50 [symbol] m. In another embodiment, the optical pathlength is about 25 [symbol] m. The windows 330, 335 are preferably formed from any of the materials discussed above as possessing sufficient radiation transmissivity. The thickness of each window is preferably as small as possible without overly weakening the sample cell 310 or cuvette 305.

Once a wound is made in the appendage 290, the opening 317 of the sample supply passage 315 of the cuvette 305 is placed in contact with the fluid that flows from the wound. In another embodiment, the sample is obtained without creating a wound, e.g. as is done with a saliva sample. In that case, the opening 317 of the sample supply passage 315 of the cuvette 305 is placed in contact with the fluid obtained without creating a wound. The fluid is then transported through the sample supply passage 315 and into the sample cell 310 via capillary action. The air vent passage 320 improves the capillary action by preventing the buildup of air pressure within the cuvette and allowing the blood to displace the air as the blood flows therein.

Other mechanisms may be employed to transport the sample to the sample cell 310. For example, wicking could be used by providing a wicking material in at least a portion of the sample supply passage 315. In another variation, wicking and capillary action could be used together to transport the sample to the sample cell 310. Membranes could also be positioned within the sample supply passage 315 to move the blood while at the same time filtering out components that might complicate the optical measurement performed by the whole-blood system 200.

FIGS. 16 and 16A depict one approach to constructing the cuvette 305. In this approach, the cuvette 305 comprises a first layer 350, a second layer 355, and a third layer 360. The second layer 355 is positioned between the first layer 350 and the third layer 360. The first layer 350 forms the first sample cell window 330 and the vent 325. As mentioned above, the vent 325 provides an escape for the air that is in the sample cell 310. While the vent 325 is shown on the first layer 350, it could also be positioned on the third layer 360, or could be a cutout in the second layer, and would then be located between the first layer 360 and the third layer 360 The third layer 360 forms the second sample cell window 335.

The second layer 355 may be formed entirely of an adhesive that joins the first and third layers 350, 360. In other embodiments, the second layer may be formed from similar materials as the first and third layers, or any other suitable material. The second layer 355 may also be formed as a carrier with an adhesive deposited on both sides thereof. The second layer 355 forms the sample supply passage 315, the air vent passage 320, and the sample cell 310. The thickness of the second layer 355 can be between about 1 [symbol] m and about 1.22 mm. This thickness can alternatively be between about 1 [symbol] m and about 100 [symbol] m. This thickness could alternatively be about 80 [symbol] m, but is preferably between about 10 [symbol] m and about 50 [symbol] m. In another embodiment, the second layer thickness is about 25 [symbol] m.

In other embodiments, the second layer 355 can be constructed as an adhesive film having a cutout portion to define the passages 315, 320, or as a cutout surrounded by adhesive.

Further information can be found in U.S. patent application Ser. No. 10/055,875, filed Jan. 21, 2002, titled REAGENT-LESS WHOLE-BLOOD GLUCOSE METER. The entire contents of this patent application are hereby incorporated by reference herein and made a part of this specification.

### II. AVOIDING CLINICALLY SIGNIFICANT FALSE READINGS

Disclosed herein are methods and apparatus for computing an estimated analyte concentration having an associated first error that is clinically significant, and processing the estimated analyte concentration to generate an adjusted analyte concentration that is accurate or has a second error that is clinically insignificant. The Clarke error grid is presented as an example of a model that may be employed to determine the clinical significance of an error. However, it should be appreciated that the apparatus and methods disclosed herein may be used in the context of any suitable classification model or technique, presently known or hereafter developed, or any subsequent updates or revisions of the Clarke error grid.

The methods disclosed herein may be employed to reduce the clinical significance of errors in measurements made by or with any analyte detection system or technique. Where it is desired to create a profile of the error-creation behavior (see further discussion below) of an analyte detection system or technique, individual measurement errors may be determined by comparing an analyte concentration measurement taken by or with the detection system(s) or technique(s) in question, with a simultaneous or near-simultaneous measurement taken via a recognized high-precision technique employing, for example, a laboratory-grade device of the type manufactured by Yellow Springs Instruments, Inc., or any other suitable high-precision device or method.

### A. Clinical Significance

The clinical significance of an erroneous measurement depends partially on the raw deviation of the measurement from the actual value. However, deviations in measurements of an analyte at a first concentration level may be clinically more significant than deviations in measurements of an analyte at a second concentration level.

As an example, the measurement of blood glucose concentrations can have a small range of acceptable errors at one concentration, while having a large range of acceptable errors at another concentration. The clinical significance of the error varies depending on the patient's actual blood glucose concentration.

In general, a patient with a glucose concentration greater than 180 mg/dL should take corrective action such as administering insulin to lower the glucose concentration. Similarly, a patient having a glucose concentration less than 70 mg/dL should take corrective action to raise the glucose concentration. When a patient has a glucose concentration in the range of 70 mg/dL to 180 mg/dL, there is generally no need to take corrective action. Reporting a glucose level of 160 mg/dL when the actual value is 140 mg/dL does not result in a clinically significant error. However, erroneously reporting a glucose level of 71 mg/dL when the actual value is 57 mg/dL can have dire clinical consequences because the patient will fail to treat the low glucose level.

While failure to treat may have serious effects upon the patient, providing the wrong treatment can be even more serious. Reporting a glucose level of 182 mg/dL when the actual glucose level is 68 mg/dL causes a patient to administer insulin when the patient should instead administer glucose.

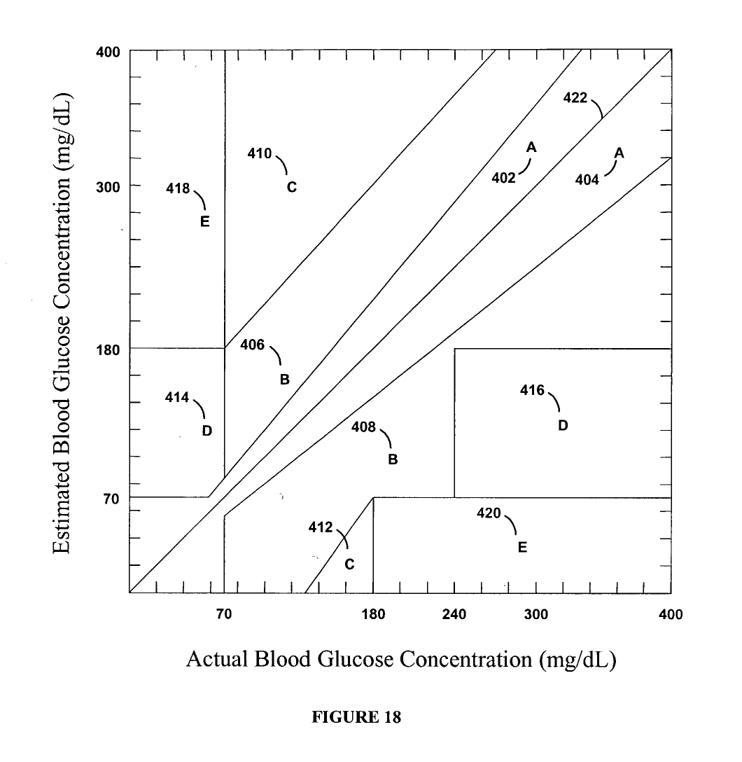

FIG. 18 classifies erroneous measurements by their clinical implications. The identity line 422 represents instrument readings that correspond exactly with the actual glucose levels. The area above the identity line 422 represents instrument readings that are higher than the actual glucose levels, and the area below the identity line 422 represents instrument readings that are lower than the actual glucose levels.

When a patient's actual glucose level is in the hypoglycemic range (for example, less than 70 mg/dL), the patient should administer glucose. Accordingly, if the instrument reports a measurement which is erroneous but nonetheless is less than or equal to 70 mg/dL, a hypoglycemic patient will still correctly administer glucose. Thus, the estimate is clinically “accurate” even though the instrument fails to report the true glucose level. For actual glucose, levels greater than 70 mg/dL, an estimated measurement is still clinically accurate as long as it deviates less than 20% from the true glucose level. The clinically accurate zones above and below the identity line 422 are referred to respectively as upper zone A 402 and lower zone A 404.

If the instrument reports an erroneous measurement that is greater than 70 mg/dL when the patient's actual glucose level is less than 70 mg/dL, the error is serious because the patient fails to administer glucose when in fact glucose is needed. Zone D 414 as shown in FIG. 18 represents erroneous measurements that cause a patient to fail to administer glucose when glucose is needed, and zone E 418 represents erroneous measurements that cause a patient to administer insulin when instead the patient should administer glucose.

Similarly, if the instrument reports an erroneous measurement that is less than 70 mg/dL when the patient's actual glucose level is greater than 180 mg/dL, the error is serious because the patient administers glucose when the patient actually needs insulin. If instead the instrument reports an erroneous measurement that is greater than 180 mg/dL when the patient's actual glucose level is less than 70 mg/dL, the patient will administer insulin when the patient really needs glucose. Both zone E 418 and zone E 420 represent serious erroneous measurements that lead a patient to administer the wrong treatment.

While not as significant as errors that cause an incorrect treatment, errors that lead to a failure to treat are also dangerous. For example, if an instrument reports an erroneous measurement that is greater than 70 mg/dL but less than 180 mg/dL when the patient's actual glucose level is greater than 240 mg/dL, the error is dangerous because the patient will fail to administer the needed insulin. Similarly if an instrument reports an erroneous measurement that is greater than 70 mg/dL but less than 180 mg/dL when the actual glucose level is less than 70 mg/dL, the error is dangerous because the patient will fail to administer the needed glucose. Zone D 414 and zone D 416 represent erroneous measurements that result in a dangerous failure to treat when treatment is needed.

Zone B 406 and zone B 408 represent erroneous measurements that are clinically neutral or benign. Zone C 410 and zone C 412 represent erroneous measurements that result in unnecessary correction of an acceptable glucose level.

The particular classification of erroneous measurements by their clinical implications shown in FIG. 18 is known as the Clarke error grid. Zone A represents estimated glucose values that deviate above or below the actual value by less than 20%, or estimated glucose values that are less than 70 mg/dL when the actual value is less than 70 mg/dL. Zone B represents values that deviate from the actual value by greater than 20%, but these deviations lead to benign or no treatment. Zones A and B are considered clinically insignificant. Zone C represents values that would result in an unnecessary correction of acceptable glucose. Zone D represents values that would result in a dangerous failure to detect and treat. Zone E represents values that would lead to treatment opposite of what clinical accuracy would call for.

It should be understood that the patent rights arising hereunder are not to be limited to the implementation of the Clarke error grid, including the specific grid depicted in FIG. 18. Medical science is a rapidly developing field, and other ways of classifying the clinical significance of erroneous measurements may currently be known or arise in the future. Additionally, the zones set out by Clarke may be adjusted by additional research. The Clarke grid provides an analysis of clinically significant errors for the general public, but does not factor in individual characteristics such as age, weight, metabolism or other characteristics unique to an individual. A grid representing clinically significant errors for a specific individual is likely to vary from the Clarke error grid. The Clarke error grid depicts the clinical significance of estimated blood glucose concentrations. However, in another embodiment the error grid is adapted to determine the clinical significance of estimated concentrations for other analytes. It is to be understood that the patent rights arising hereunder are not to be limited to the specific embodiments or methods described in this specification or illustrated in the drawings, but extend to other arrangements, technology, and methods, now existing or hereinafter arising, which are suitable or sufficient for achieving the purposes and advantages hereof.

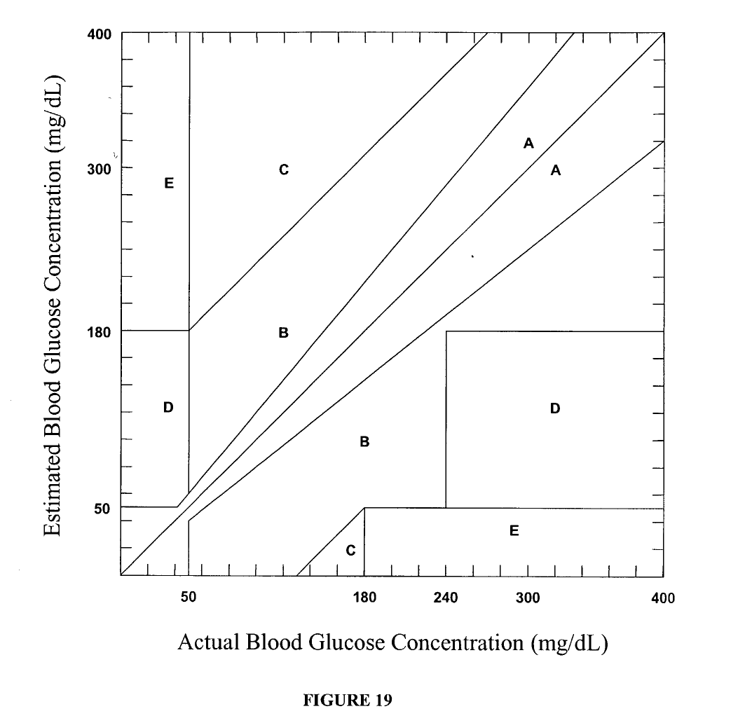

The clinical significance of a measurement is partially dependent upon a determination of where the clinical treatment constraints should lie. For example, the threshold of clinical significance for blood glucose measurements of a hypoglycemic patient could be at 70 mg/dL as depicted in FIG. 18, or the threshold could be at other points such as 50 mg/dL or 60 mg/dL. FIG. 19 depicts a grid where the threshold of clinical significance for a hypoglycemic patient is at 50 mg/dL. For convenience, the following embodiments generally refer to the Clarke error grid as depicted in FIG. 18 having a threshold of clinical significance at 70 mg/dL; however, it will be appreciated that these embodiments are equally applicable where such threshold is located at any other suitable concentration level.

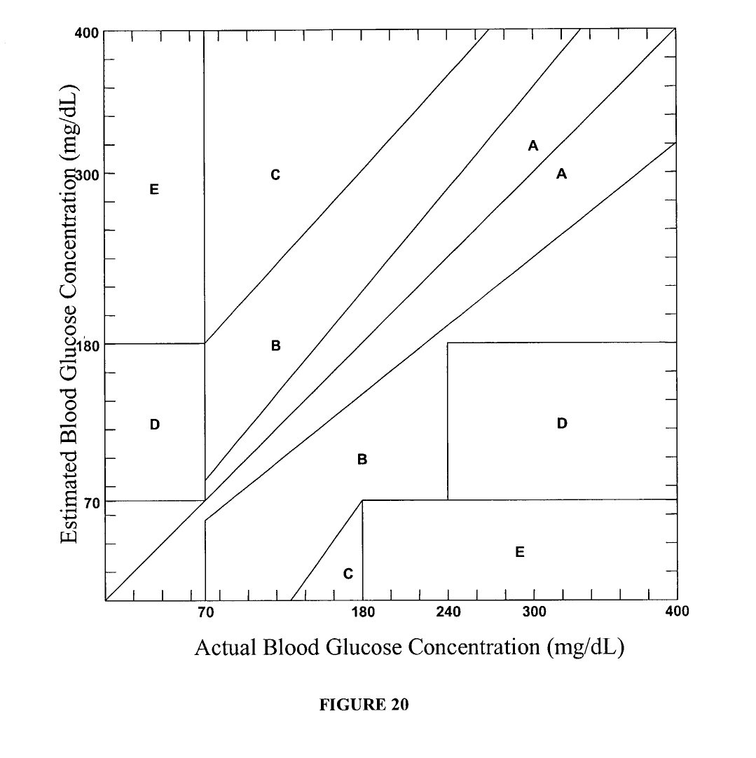

It is contemplated that the embodiments set forth herein are applicable to any revisions to the Clarke error grid, with appropriate adjustments made in the functioning to accommodate changes in the grid. For example, FIG. 20 depicts a grid where zone D extends all the way to the identity line. Using this modified grid, reporting any blood glucose concentration above 70 mg/dL when the actual blood glucose concentration is less than 70 mg/dL is clinically significant. This differs from the grid illustrated in FIG. 18, where a measurement is clinically significant when the actual, blood glucose concentration is less than 70 mg/dL, but the measured blood glucose concentration is greater than 70 mg/dL and also greater than 120% of the reference concentration.

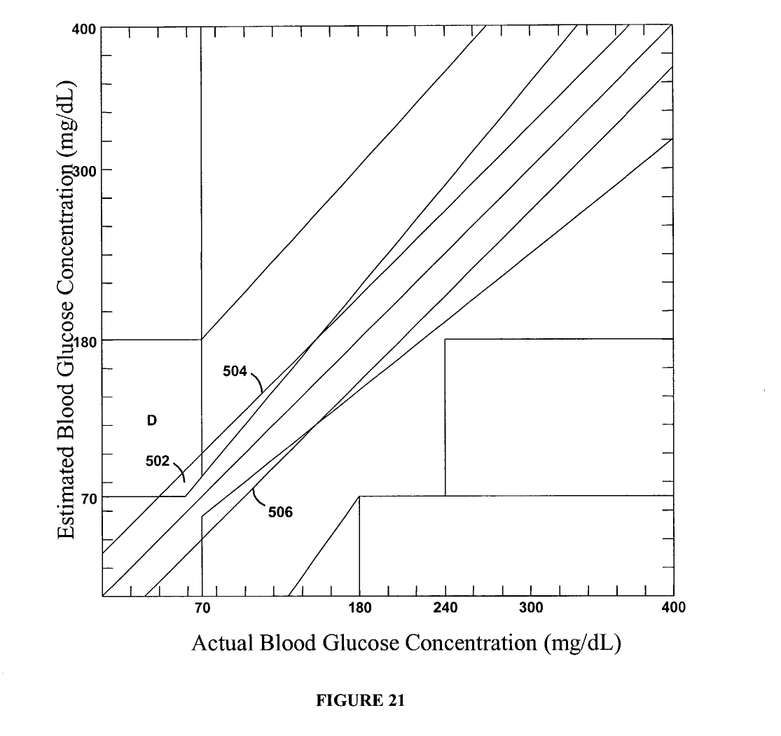

Many of the clinically significant errors as illustrated by the Clarke error grid are not likely to occur. For example, the probability of an error having a given magnitude may be known for an instrument. FIG. 21 depicts potential errors for an instrument that is known to provide an estimated blood glucose concentration within ±30 mg/dL of the actual blood glucose concentration. The line 504 represents a deviation of +30 mg/dL from the identity line, and the line 506 represents a deviation of −30 mg/dL from the identity line. There is a potential for reporting erroneous values that are clinically significant, as illustrated by the area 502. Thus, an estimated blood glucose concentration that falls between 70 mg/dL and 100 mg/dL as indicated by the vertical axis might result in a clinically significant error.

For convenience, several of the following embodiments refer to an instrument that provides an estimated blood glucose concentration within ±30 mg/dL of the actual blood glucose concentration. However, an instrument may be accurate within other ranges, such as ±25 mg/dL, ±20 mg/dL, ±15 mg/dL, or ±10 mg/dL. Additionally, an instrument may be more accurate at estimating some concentrations and less accurate at estimating other concentrations. In another example, the accuracy may be related to the concentration of the analyte. For example, an instrument may provide a measurement that is within ±20% of the true analyte concentration. It is to be understood that the exemplary use of ±30 mg/dL is not to be taken as limiting, because the methods and devices disclosed herein are applicable with a wide variety of error bands.

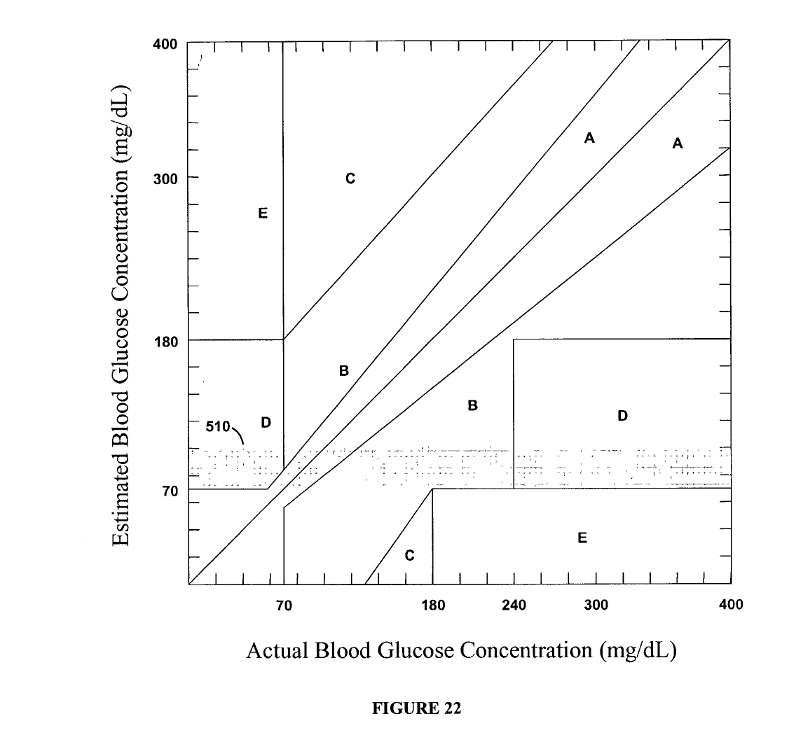

An instrument that estimates a blood glucose concentration may not “know” if the measurement is correct, or if the measurement deviates from the actual value. FIG. 22 illustrates a band 510 that represents estimated values falling between 70 mg/dL and 100 mg/dL. An estimated value that falls into the band 510 is a clinically significant error if the patient's actual value is less than 70 mg/dL or greater than 240 mg/dL. Assuming that the instrument is known to be accurate within ±30 mg/dL, it is highly unlikely or impossible that the instrument will report an estimate between 70 and 100 mg/dL when the patient's actual value is 240 mg/dL or more. Therefore, the concern is that, where the patient has an actual blood glucose concentration between 40 mg/dL and 70 mg/dL, the instrument might report an estimate between 70 mg/dL and 100 mg/dL, which would result in a clinically significant error.

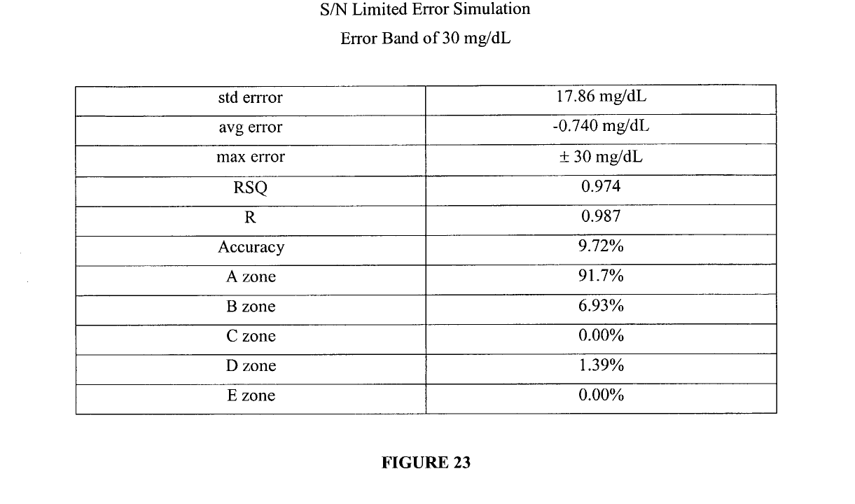

FIG. 23 shows the results of a simulation that models a theoretical blood analyte detection instrument having a maximum error deviation of ±30 mg/dL. Using a random number generator and a statistical model, 361 sample points were generated. The error of the sample points had a standard deviation of 17.86 mg/dL, and 98.6% of the sample points were in the clinically acceptable zones A and B of the Clarke error grid. However, 1.4% of the sample points were in the clinically “dangerous” zone D. The sample points in zone D could lead a diabetic to fail to administer glucose when glucose is needed. This example shows the dangers of using an instrument that provides erroneous readings leading to the mistreatment of a patient.

With a good instrument, most erroneous readings will fall within zone A. It can be observed from the results shown in FIG. 23 that most erroneous readings not in zone A fall into the adjacent zone B. However, zone D adjoins the upper edge of zone A near readings of 70 mg/dL, so erroneous high readings in this area are critical.

The results shown in FIG. 23 are reported using standard statistical nomenclature. Definitions for common statistical terms used in the analysis are set forth below.

Correlation Coefficient R: A standard statistical measure of the precision of a least-squares linear fit between two random variables. For random variables x and y, R (x,y) is a number between −1 and 1, calculated as the ratio of the covariance κ (x,y) divided by the product of the individual standard deviations σₓ,σ_{y} of the variables. A correlation coefficient R of ±1 implies an ideal linear relation between x and y.

Covariance κ: A standard statistical measure of a least-squares linear fit between two random variables x and y. κ (x,y) is computed as the mean value of the product (x−\overline{x})(y−\overline{y}) where \overline{x}, \overline{y} denote the mean values of x and y, respectively.

For a set of N observations {x₁,x₂, . . . ,x_{N}} and {y₁,y₂, . . . ,y_{N}}, the covariance is:

$$\frac{1}{N - 1} {\sum }_{i = 1}^{N} ( {x}_{i} - \overline{x} ) ( {y}_{i} - \overline{y} )$$

Mean: The statistical average of a random variable x. For a set of N observations {x₁,x₂, . . . ,x_{N}}, the sample mean \overline{x} is given by:

$$\frac{1}{N} {\sum }_{i = 1}^{N} {x}_{i}$$

Standard Deviation σ: A standard statistical measure of the spread of a random variable about its mean value. For a set of N observations {x₁,x₂, . . . ,x_{N}} the sample standard deviation is given by:

$${σ}_{x} = \sqrt{\frac{1}{N - 1} {\sum }_{i = 1}^{N} {( {x}_{i} - \overline{x} )}^{2}}$$

The standard deviation is also called the standard error of the random variable. For gaussian variables, approximately 68% of observations will fall within one standard deviation of the mean value. For instance, the standard error of a model ŷ for the random variable y is the standard deviation of the model error ε=ŷ−y, i.e. σ(ŷ−y).

Variance V. A standard statistical measure of the spread of a random variable about its mean value. For a set of N observations {x₁,x₂, . . . ,x_{N}), the sample variance

$${v}_{x} = \frac{1}{N - 1} {\sum }_{i = 1}^{N} {( {x}_{i} - \overline{x} )}^{2} ,$$

where \overline{x} is the sample mean. Variance is related to the correlation coefficient as follows: if x and y are random variables having a correlation coefficient of R, then the least-squares linear fit of y using x (i.e., ŷ=mx+b is a linear estimator of y) will enable the model to explain R² of the variance of y by variation of the x variable. For instance, an R² value of 0.80 provides a model that accounts for 80% of the variation in y, using x.

### B. Error Band

Any instrument that performs analysis such as the noninvasive system 10 or the whole-blood system 200 has an error band associated with it. Factors that contribute to the error band may come from the instrument itself or from the environment in which the instrument operates. Manufacturers often know the worst case error band of their instruments.

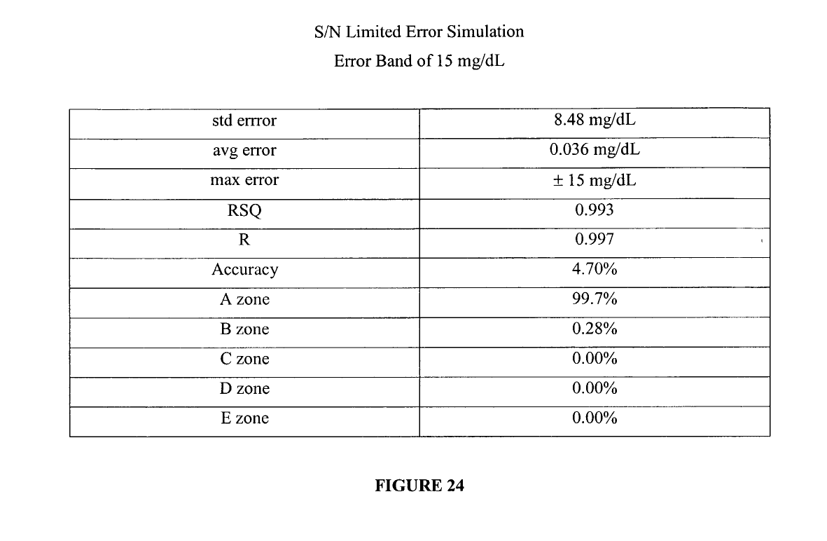

Even though a good instrument can have a low standard error, statistically there will still be a small percentage of errors that cause readings to occur in zone D. FIG. 24 illustrates a clinical data set from a hypothetical instrument with a narrow error band of 15 mg/dL. This simulation represents a current “state of the art” instrument with a low error rate.

In general, the accuracy of an instrument or method may be determined by comparing measurements taken therewith against simultaneous or near-simultaneous measurements taken via a known high-precision technique. More specifically, measurements taken with a system such as, but not limited to, the non-invasive system 10 or the whole blood system 200, may be compared against measurements taken with, for example, a laboratory-grade device of the type manufactured by Yellow Springs Instruments, Inc., or any other suitable instrument to determine any error in the measurement taken with the system or method in question.

In one embodiment, the magnitude of the error band is the maximum expected or observed deviation from identity. The magnitude of the error band may thus, in one embodiment, be the absolute value of the largest error observed in a number of test measurements performed with the analyte detection system or technique in question. For example, assume that a suitably large number of test measurements are first taken with the instrument/technique, and compared against corresponding measurements taken with a known high-precision technique as discussed above. From these test measurements, it is found that the largest observed error is 20 mg/dL (e.g., a value of 100 mg/dL (or 60 mg/dL) is reported when the actual value is 80 mg/dL). An error band of 20 mg/dL is therefore a suitable choice for this instrument/technique. Alternatively, the error band can be defined by employing any suitable technique or statistical analysis for estimating or determining the maximum expected or observed error of the analyte detection system in question.

For purposes of illustration, assume that statistical analysis of the error behavior of the instrument/technique shows that the largest expected error is 30 mg/dL. A selection of an error band of 30 mg/dL is appropriate for this instrument or technique.

In another embodiment, the error band can be computed as the sum of the mean error observed in a set of test measurements, compiled as discussed above, plus some multiple of the standard deviation of the observed error.

For example, given a mean error \overline{x}, the error band can be \overline{x}+1.0·σ, \overline{x}+2.0·σ, or the sum of \overline{x} and any suitable integer or decimal multiple of σ.

For purposes of illustration, assume that observation of test measurements from an instrument or method reveals that the mean error \overline{x} is 0.2 mg/dL and the standard deviation σ of the error is 5.0 mg/dL. A desired error band of \overline{x}+2.5 σ thus has a magnitude of 12.7 mg/dL.

In another embodiment, the error band is chosen to encompass a selected percentage of observed or expected errors (or, by extension, analyte-concentration measurements), such as 80%, 85%, 90%, 95%, 99%, 99.9%, 99.99% or any other suitable percentage. For example, the error band magnitude could be selected to be greater than or equal to (or “encompass”) 99.9% of the observed or expected errors or measurements.

As an illustration, assume that a statistical analysis reveals that 99.9% of the observed or expected errors are 30 mg/dL or less. Selecting the error band to have a magnitude of 30 mg/dL means that the error band encompasses 99.9% of the errors, and 99.9% of the analyte-concentration measurements will fall no farther than 30 mg/dL from identity.

Where the error profile of an instrument or technique appears to be asymmetric (for example, where the average positive deviation from identity is larger than the average negative deviation from identity, or vice versa) it may be desirable to calculate two separate error bands, one calculated from the positive deviations and another calculated from the negative deviations. As one example a positive error band is calculated as 25 mg/dL above identity, while a negative error band is calculated as 15 mg/dL below identity. Caution may necessitate using the larger of the two error bands in any of the methods discussed below for adjusting raw analyte concentration measurements. Alternatively, the value of the error band closest to a clinically significant zone may be selected. For example, the magnitude of the positive error band could be used to avoid Zone D 414 as illustrated in FIG. 18.

Where desired, the error band may be increased, after initial calculation, by a suitable safety margin.

### C. Avoiding Clinically Significant Errors

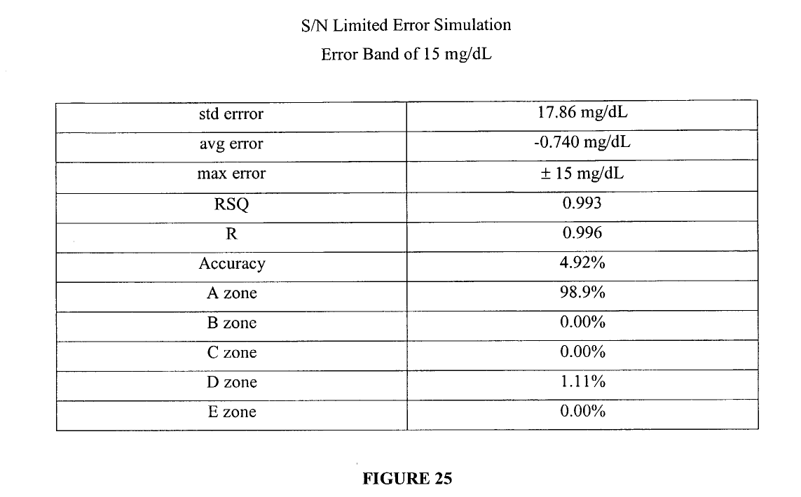

FIG. 24 shows the results of a simulation where 100% of the data points fall in zones A and B and none of the points fall in zone D. However, as shown in FIG. 25, running the random simulation again with the same parameters resulted in four of the samples falling into the dangerous zone D.

The statistical nature of the errors associated with glucose measurement make it difficult to prevent erroneous readings that fall into zone D. Even though random readings that fall into zone D are not common, they do still occur and a single reading might not reveal the error. Most patients only take a single measurement to determine blood glucose levels, so unless the patient suspects the reading is in error and takes a second measurement the erroneous reading is likely to go unexposed until it is too late.

While it may not be possible to completely eliminate erroneous readings, the clinical significance of those erroneous readings can be minimized. As stated previously, erroneous measurements falling into zone A are clinically accurate. Erroneous measurements falling into zone B are clinically neutral or benign errors. One method of avoiding an incorrect treatment is to display an adjusted glucose level so that the displayed measurements tend to fall into zone A or zone B instead of zone D or zone E.

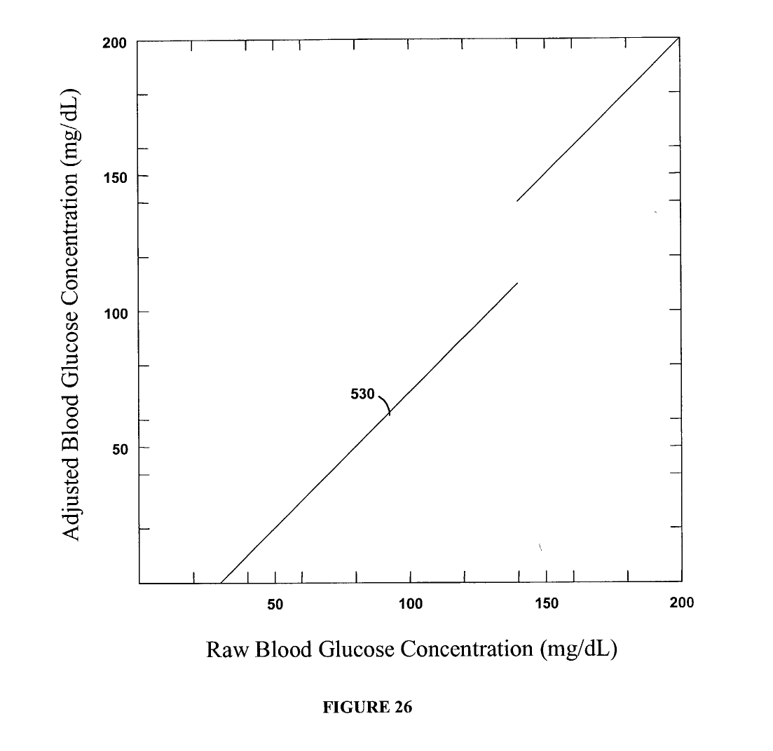

FIGS. 26 and 29-33 illustrate graphs of functions for adjusting glucose levels to avoid clinically significant errors. The graphs plot a Raw Blood Glucose Concentration measurement (or “raw measurement”) on the horizontal axis and an Adjusted Blood Glucose Concentration measurement (or “adjusted measurement”) on the vertical axis. The raw measurement is adjusted according to a transfer function (embodiments of which are discussed in detail below) to generate a corresponding adjusted measurement.

FIG. 26 illustrates one embodiment of a transfer function 530. The illustrated transfer function 530 is characterized in that adjusted measurements greater than 140 mg/dL are identical to the raw measurements performed by the instrument. The transfer function 530 adjusts raw measurements below or equal to 140 mg/dL to generate glucose levels which are, for example, one error band lower than the raw measurements. In the example illustrated in FIG. 26, the raw measurements are adjusted by 30 mg/dL. The adjustment of raw measurements results in more benign zone B errors, but reduces the number of dangerous zone D errors.

In the example illustrated in FIG. 26, an instrument having an error band of 30 mg/dL takes a measurement of a patient with an actual glucose level of 60 mg/dL. If the instrument provides a raw measurement of 80 mg/dL, this erroneous measurement falls into zone D (see FIG. 18). In this situation, if the patient takes the 80 mg/dL raw measurement “at face value” he or she will decline to administer glucose when in fact glucose is needed. This zone D error (defined as a dangerous failure to treat) becomes more serious if the patient administers a wrong treatment. Faced with an incorrect raw measurement of 80 mg/dL glucose level, a patient might self-administer insulin. Administering insulin under conditions of an actual 60 mg/dL glucose level can be highly dangerous. However, by applying the transfer function 530, the displayed measurement is adjusted by one error band lower than the raw measurement, and the instrument displays a result of 50 mg/dL. Although the displayed measurement of 50 mg/dL is in error, it is now a clinically benign zone A error. Given a reading of 50 mg/dL, the patient will administer glucose, which is the correct treatment decision. In other words, the 50 mg/dL displayed measurement generated by the transfer function 530 is a clinically accurate estimate.

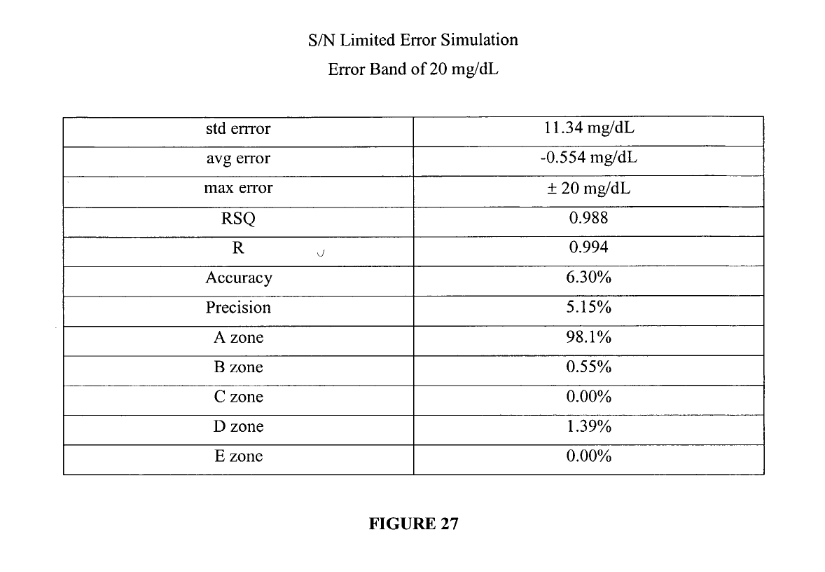

FIG. 27 shows a set of raw measurements obtained from a simulation of an instrument having an error band of 20 mg/dL. An error band of 20 mg/dL is slightly worse than the typical 15 mg/dL error band for a state of the art instrument. A total of 361 sample points were generated, with five of the raw measurements falling in zone D, and two of the raw measurements falling in zone B. The remaining measurements fell in zone A.

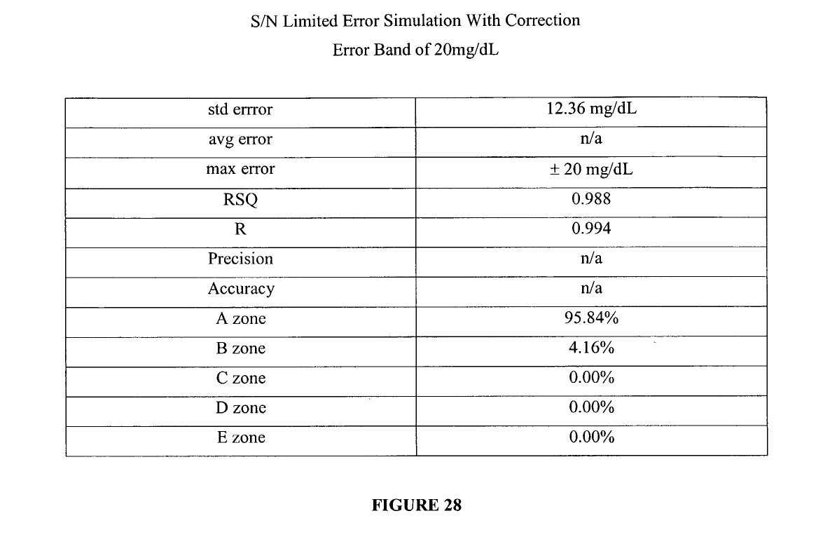

FIG. 28 shows an example of a set of adjusted measurements obtained with a simulated instrument that utilizes a transfer function to avoid readings that fall into zone D. The simulated instrument applied a transfer function to the raw measurements obtained from the simulation shown in FIG. 27. Most of the data points shown in FIG. 28 are equivalent to those shown in FIG. 27. However, where zone D adjoins zone A near 70 mg/dL, the adjusted measurements in FIG. 28 are lower than the raw measurements in FIG. 27.

Applying the transfer function does not significantly alter the R value, which correlates the displayed measurements and the actual blood glucose levels. The transfer function does raise the number of zone B errors from 2 to 15. However, zone B errors are not clinically significant. Using the transfer function, all of the zone D errors are eliminated and 100% of the data points fall into zones A and B.

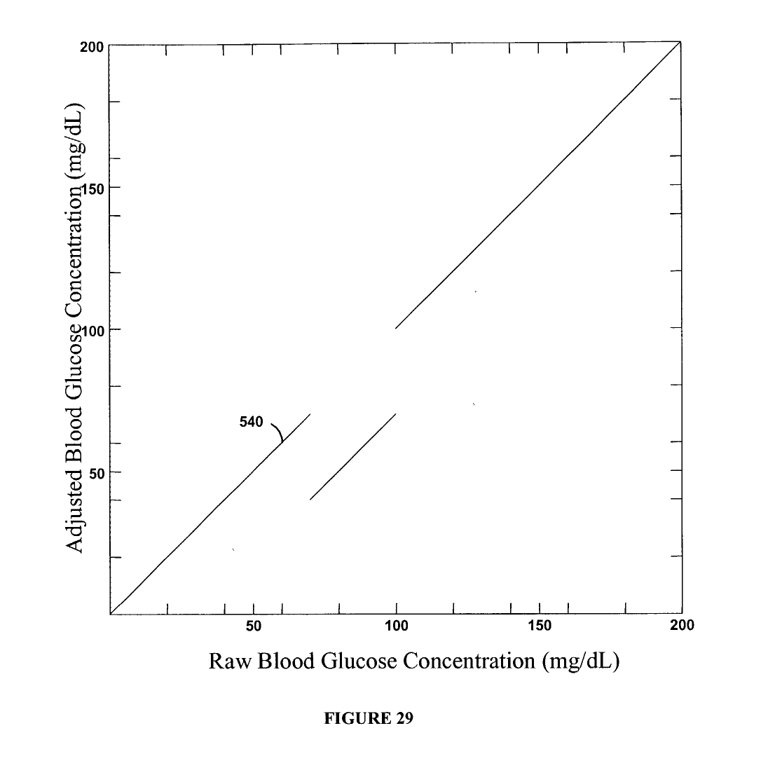

FIG. 29 depicts another embodiment of a transfer function 540. The transfer function 540 returns adjusted measurements that are lower than the raw measurements (by one error band; in this example 30 mg/dL) when the raw measurements are greater than or equal to 70 mg/dL and less than or equal to 100 mg/dL. For other raw measurements, the transfer function 540 returns adjusted measurements that are equal to the raw measurements.

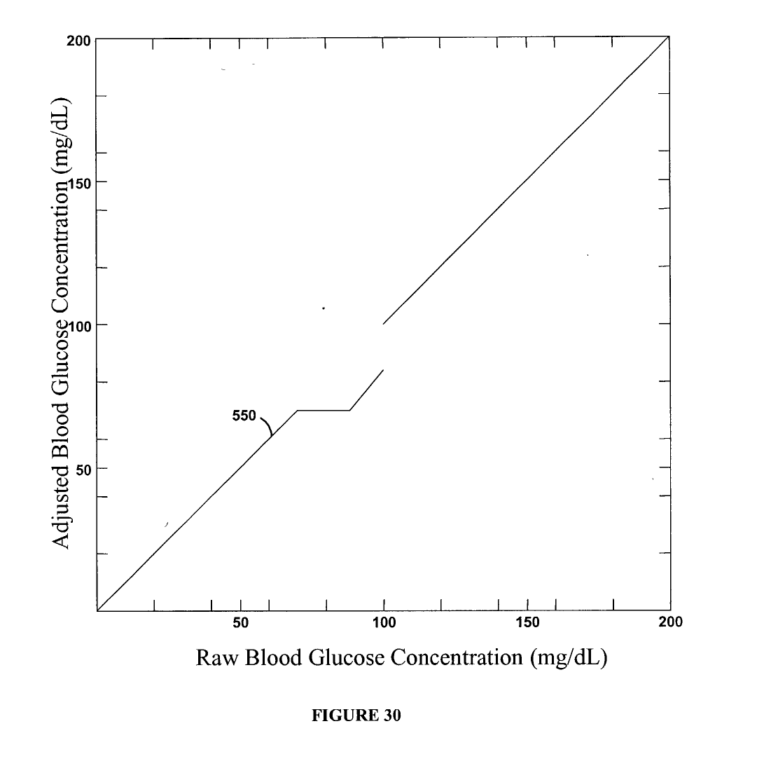

FIG. 30 depicts another embodiment of a transfer function that advantageously minimizes the effect that adjusting measurements has on the R value. Consider, for example, an instrument having a maximum error of 30 mg/dL that obtains a raw measurement of 100 mg/dL. Given the possible error of the instrument, the actual concentration will be somewhere between 70 mg/dL and 130 mg/dL. Referring to FIG. 18, obtaining an erroneous measurement of 100 mg/dL means that the error is clinically significant only if the actual concentration is 70 mg/dL, which places the erroneous measurement on the edge of zone D 414. However, if the instrument reported an adjusted measurement below 84 mg/dL for an actual value of 70 mg/dL, the error is no longer clinically significant. While any value below 84 mg/dL could be selected to avoid a clinically significant error, the effect on the R value is minimized by selected an adjusted measurement of 84 mg/dL.

If the same instrument were to obtain a raw measurement of 85 mg/dL, the actual concentration would be somewhere between 55 mg/dL and 115 mg/dL. Referring again to FIG. 18, obtaining an erroneous measurement of 85 mg/dL means that the error is clinically significant only if the actual concentration is between 55 mg/dL and 70 mg/dL. While any value below 70 mg/dL could be selected to avoid a clinically significant error, the effect on the R value is minimized by selecting an adjusted measurement of 70 mg/dL.

FIG. 30 depicts an embodiment of a transfer function 550 that adjusts raw measurements by a minimum value while still avoiding erroneous measurements that fall into zone D. Assuming an instrument has an error band of 30 mg/dL, raw measurements having a value less than or equal to 70 mg/dL or greater than 100 mg/dL are not adjusted. Raw measurements having a value greater than 70 mg/dL and less than or equal to 100 mg/dL could potentially result in clinically significant errors.

However, to avoid clinically significant errors an instrument needs to adjust the raw measurement only by an amount sufficient to remove erroneous measurements from zone D. Thus, converting raw measurements that are greater than 70 mg/dL and less than 88 mg/dL into an adjusted measurement of 70 mg/dL places the adjusted measurements on the lower border of zone D. As shown in FIG. 18, the lower border of zone D follows a line corresponding to 120% of the identity line for actual concentrations from 58 mg/dL to 70 mg/dL. As the instrument in this example has an error band of 30 mg/dL, an actual concentration of 58 mg/dL could have a worst case raw measurement of 88 mg/dL. Adjusting raw measurements greater than or equal to 88 mg/dL and less than or equal to 100 mg/dL to have an adjusted measurement that falls on the sloping border of zone D minimizes the effect of the adjustment on the R value. Thus, the transfer function defined below and illustrated in FIG. 30 avoids clinically significant errors while minimizing the effect caused by adjusting measurements.

$${m}_{a} = \{ \begin{matrix} {m}_{r} \\ 1.2 \cdot ( {m}_{r} - 30 mg / dL ) \\ 70 mg / dL \\ {m}_{r}\end{matrix} \begin{matrix} 100 mg / dL < {m}_{r} \\ ( \frac{70}{1.2} + 30 ) mg / dL < {m}_{r} \leq 100 mg / dL \\ 70 mg / dL < {m}_{r} \leq ( \frac{70}{1.2} + 30 ) mg / dL \\ {m}_{r} \leq 70 mg / dL\end{matrix} \\ {m}_{r} = raw measurement \\ {m}_{a} = adjusted measurement$$

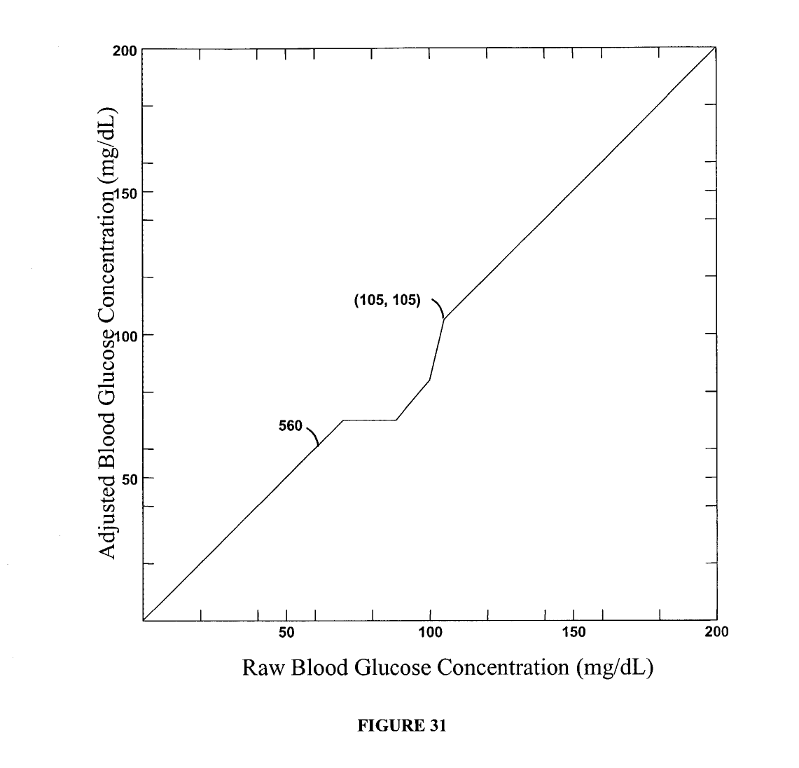

FIG. 31 depicts an embodiment of a transfer function 560 that is similar in most respects to the transfer function 550 depicted in FIG. 30. However, the transfer function 560 tapers from a point at which a raw measurement of 100 mg/dL is converted into an adjusted measurement of 85 mg/dL to a point at which a raw measurement of 105 mg/dL is converted into an adjusted measurement of 105 mg/dL. Tapering is advantageous where a patient takes multiple readings, because the tapered transfer function does not cause an abrupt change between adjusted readings based on closely spaced raw measurements. Referring back to FIG. 30, a patient taking a first reading where the raw measurement is 101 mg/dL will see an adjusted measurement of 101 mg/dL. If the patient takes a second reading and the raw measurement is 100 mg/dL, the patient will see an adjusted measurement of 84 mg/dL. The abrupt change in readings may cause concern to the patient. However, if the transfer function gradually tapers in its conversion of raw measurements to adjusted measurements as seen in FIG. 31 and defined below, the patient will not see a sudden jump between readings.

$${m}_{a} = \{ \begin{matrix} {m}_{r} \\ \frac{10}{3} \cdot {m}_{r} - 245 mg / dL \\ 1.2 \cdot ( {m}_{r} - 30 mg / dL ) \\ 70 mg / dL \\ {m}_{r}\end{matrix} \begin{matrix} 105 mg / dL < {m}_{r} \\ 100 mg / dL < {m}_{r} \leq 105 mg / dL \\ ( \frac{70}{1.2} + 30 ) mg / dL < {m}_{r} \leq 100 mg / dL \\ 70 mg / dL < {m}_{r} \leq ( \frac{70}{1.2} + 30 ) mg / dL \\ {m}_{r} \leq 70 mg / dL\end{matrix} \\ {m}_{r} = raw measurement \\ {m}_{a} = adjusted measurement$$

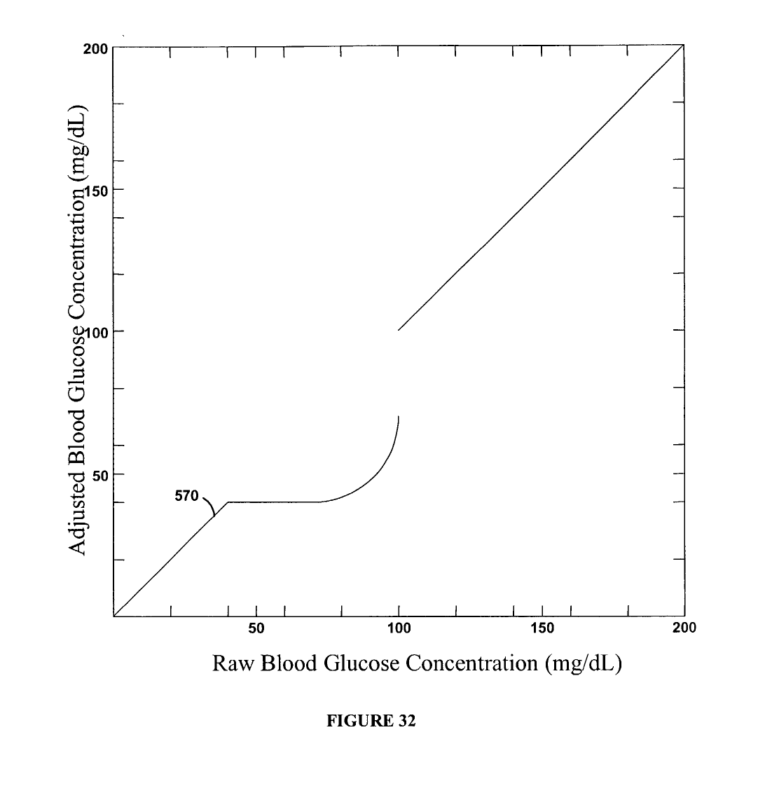

FIG. 32 depicts an embodiment of a transfer function 570 that converts raw measurements greater than or equal to 0 mg/dL and less than, 40 mg/dL into adjusted measurements that are equal to the raw measurements. For raw measurements that are greater than or equal to 40 mg/dL and less than 70 mg/dL, the transfer function 570 returns adjusted measurements that are one error band (in this example, 30 mg/dL) below the lowest portion of zone D 414 (see FIG. 18), typically a horizontal line located at 70 mg/dL. For raw measurements that are greater than or equal to 70 mg/dL and less than or equal to 100 mg/dL, the transfer function 570 returns adjusted measurements defined by an arc having a radius of 30 and a center on the identity line at 70 mg/dL. The transfer function 570 converts raw measurements greater than 100 mg/dL into adjusted measurements that are equal to the raw measurements.

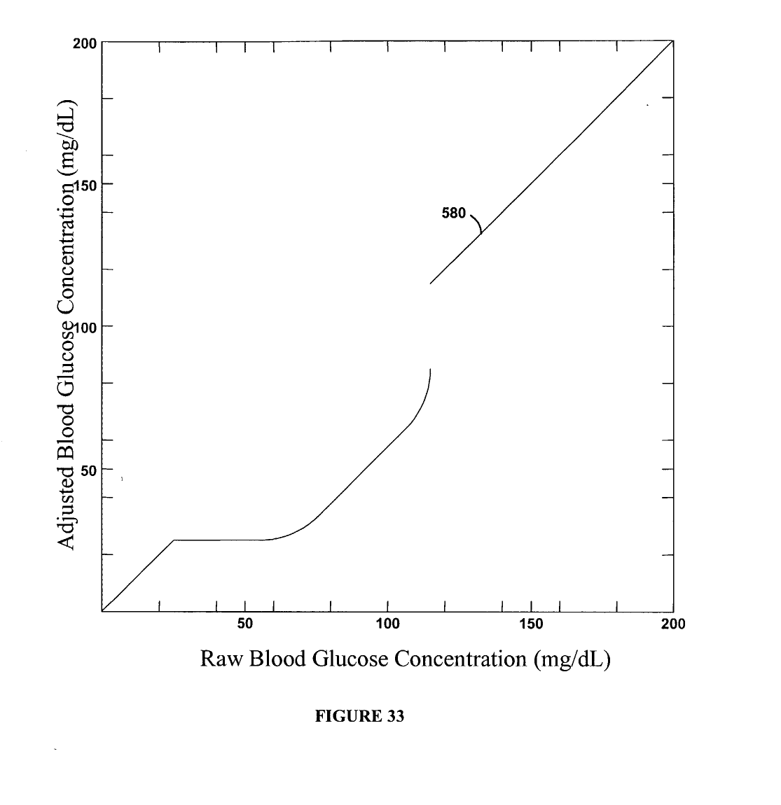

FIG. 33 depicts an embodiment of a transfer function 580 that converts raw measurements greater than or equal to 0 mg/dL and less than 25 mg/dL into adjusted measurements that are equal to the raw measurements. For raw measurements that are greater than or equal to 25 mg/dL and less than 55 mg/dL, the transfer function 580 returns adjusted measurements of 25 mg/dL. For raw measurements that are greater than or equal to 55 mg/dL and less than 76 mg/dL, the transfer function 580 returns adjusted measurements defined by an arc having a radius of 30 and a center on the identity line at 55 mg/dL. For raw measurements that are greater than or equal to 76 mg/dL and less than 106 mg/dL, the transfer function 580 returns adjusted measurements of that are 30 mg/dL less than the raw measurements. For raw measurements that are greater than or equal to 106 mg/dL and less than or equal to 115 mg/dL, the transfer function 580 returns adjusted measurements defined by an arc having a radius of 30 and a center on the identity line at 85 mg/dL. The transfer function 580 converts raw measurements greater than 115 mg/dL into adjusted measurements that are equal to the raw measurements.

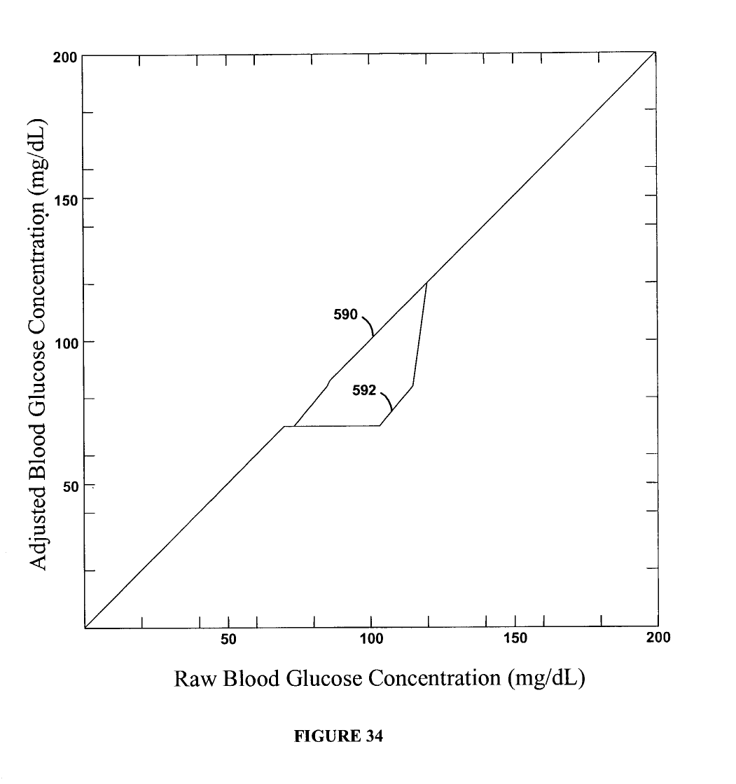

Implementation of the disclosed embodiments is not limited to using the error band of the instrument for determining an appropriate transfer function. Any of the above described embodiments can be modified by applying adjustments greater or smaller than the instrument's error band where appropriate. For example, FIG. 34 depicts transfer functions 590 and 592 that are generally similar to the function 560 depicted in FIG. 31 but allow for variation in the acceptable tolerance of errors. The transfer function 590, as defined below, prevents erroneous raw measurements having a maximum deviation of 15 mg/dL from falling into zone D.

$${m}_{a} = \{ \begin{matrix} {m}_{r} \\ 2 \cdot {m}_{r} - 86 mg / dL \\ 1.2 \cdot ( {m}_{r} - 15 mg / dL ) \\ 70 mg / dL \\ {m}_{r}\end{matrix} \begin{matrix} 86 mg / dL < {m}_{r} \\ 85 mg / dL < {m}_{r} \leq 86 mg / dL \\ ( \frac{70}{1.2} + 15 ) mg / dL < {m}_{r} \leq 85 mg / dL \\ 70 mg / dL < {m}_{r} \leq ( \frac{70}{1.2} + 15 ) mg / dL \\ {m}_{r} \leq 70 mg / dL\end{matrix} \\ {m}_{r} = raw measurement \\ {m}_{a} = adjusted measurement$$

The transfer function 592, as defined below, prevents erroneous raw measurements having a maximum deviation of 45 mg/dL from falling into zone D.

$${m}_{a} = \{ \begin{matrix} {m}_{r} \\ 7.2 \cdot {m}_{r} - 744 mg / dL \\ 1.2 \cdot ( {m}_{r} - 45 mg / dL ) \\ 70 mg / dL \\ {m}_{r}\end{matrix} \begin{matrix} 120 mg / dL < {m}_{r} \\ 115 mg / dL < {m}_{r} \leq 120 mg / dL \\ ( \frac{70}{1.2} + 45 ) mg / dL < {m}_{r} \leq 115 mg / dL \\ 70 mg / dL < {m}_{r} \leq ( \frac{70}{1.2} + 45 ) mg / dL \\ {m}_{r} \leq 70 mg / dL\end{matrix} \\ {m}_{r} = raw measurement \\ {m}_{a} = adjusted measurement$$

It should be farther understood that any transfer function may be employed that generally follows the identity line of the Clarke error grid but causes raw measurements within an error band of zone D to deviate away from zone D. Given a threshold concentration (TC) above which an erroneous measurement is potentially clinically significant, a preferred embodiment adjusts raw measurements to provide an adjusted measurement below the threshold concentration. A basic transfer function could be as simple as the equation below.

$${m}_{a} = \{ \begin{matrix} {m}_{r} \\ TC \\ {m}_{r}\end{matrix} \begin{matrix} TC + Error Band < {m}_{r} \\ TC < {m}_{r} \leq TC + Error Band \\ {m}_{r} \leq TC\end{matrix} \\ {m}_{r} = raw measurement \\ {m}_{a} = adjusted measurement$$

Alternatively, other correction factors may be used as shown in the equation below. Of course, adjusting raw measurements less than or equal to the threshold concentration or greater than the threshold concentration plus the error band is also feasible.

$${m}_{a} = \{ \begin{matrix} {m}_{r} \\ {m}_{r} \\ {m}_{r}\end{matrix} - Correction Factor \begin{matrix} TC + Error Band < {m}_{r} \\ TC < {m}_{r} \leq TC + Error Band \\ {m}_{r} \leq TC\end{matrix} \\ {m}_{r} = raw measurement \\ {m}_{a} = adjusted measurement$$

In another embodiment, an instrument may use raw measurements taken over a period of time in the determination of the adjusted measurement. A filter provides an example of a way to use raw measurements taken over a period of time to determine an adjusted measurement.

Either analog or digital filters may be used. In one embodiment, the raw measurement is used as a filter input. The filter can derive the adjusted measurement from the filter inputs, or the filter can use feedback together with the filter inputs. For example, the adjusted measurements provide one type of signal that is appropriate for feedback into the filter.

The inputs to the filter may be provided continuously, or at other intervals such as milliseconds, seconds, minutes, or even hours. Although the sample intervals can be periodic, filters can also be designed to use non-periodic inputs. For example, measurements taken by a patient every few hours are not likely to be strictly periodic. An instrument can use the time between measurements as one of the filter parameters.

For some instruments, it is possible to reduce the error band, for example, by taking additional samples or increasing the sample time. This may result in additional processing requirements and slower response time. Thus, it may not be desirable to reduce the error band for every measurement. However, it may be desirable to reduce the error band when a raw measurement is near zone D.

In one embodiment, an instrument reduces the error band when a raw measurement could potentially result in a clinically significant error. For example, when an instrument that normally has an error band of 30 mg/dL determines that a raw measurement is greater than or equal to 70 mg/dL and less than or equal to 100 mg/dL, the instrument reduces the error band and obtains a more accurate raw measurement by taking additional measurements and/or increasing the measurement or analysis time. If the more accurate raw measurement could still potentially cause a clinically significant error, a transfer function such as one of the transfer functions previously described is applied to the raw measurement.

It is to be understood that while 30 mg/dL has been used for illustrative purposes of an error band, each instrument will have its own error band. The error band may be constant across the range of measurements, or the magnitude of possible errors may change as the concentration of the analyte changes. Additionally, measurements other than the error band may also be appropriate for adjusting the measured values. It is to be understood that the patent rights arising hereunder are not to be limited to the specific embodiments or methods described in this specification or illustrated in the drawings, but extend to other arrangements, technology, and methods, now existing or hereinafter arising, which are suitable or sufficient for achieving the purposes and advantages hereof.

In one embodiment, the transfer function is implemented in a non-invasive blood glucose monitor such as the non-invasive system 10 disclosed above. In another embodiment, the transfer function is implemented in a reagentless whole-blood detection system such as the whole-blood detection system 200 disclosed above. Where employed in the noninvasive system 10 or the whole-blood system 200, the transfer function could reside as a data processing algorithm or program instructions within memory accessible by the signal processor 74/260. It is contemplated that the signal processor 74/260 executes the algorithm/program containing the transfer function to convert raw measurements into adjusted measurements as disclosed in various embodiments above. Alternatively, the transfer function may be implemented as such an algorithm or program in any other suitable system for measuring analyte concentrations, presently known or hereafter developed.

It is contemplated that the methods disclosed herein can be used to improve the accuracy of a wide variety of devices, such as but not limited to detectors which analyze the constituents of blood withdrawn from a patient; noninvasive measurement devices of any type, including thermal gradient spectrometers of the type disclosed herein or in the above-mentioned U.S. Pat. No. 6,198,949 or U.S. patent application Ser. No. 09/538,164; implantable and/or subcutaneous measurement devices; devices which measure glucose levels continuously and devices which measure glucose levels intermittently. In a presently preferred embodiment, the disclosed methods are used with a thermal gradient spectrometer to increase the accuracy of its measurement of the concentration of glucose in the bodily fluids of a patient.

The specific embodiments described herein are merely illustrative. Although described in terms of certain preferred embodiments, other embodiments that are apparent to those of ordinary skill in the art, including embodiments which do not provide all of the benefits and features set forth herein, are also within the scope of this invention.

Accordingly, it is to be understood that the patent rights arising hereunder are not to be limited to the specific embodiments or methods described in this specification or illustrated in the drawings, but extend to other arrangements, technology, and methods, now existing or hereinafter arising, which are suitable or sufficient for achieving the purposes and advantages hereof.

What is claimed is:

1. A blood glucose detection system comprising a processing circuit which:
identifies possible zone D errors among estimated blood glucose concentration values; and
converts those of said estimated blood glucose concentration values which are identified as possible zone D errors, into adjusted blood glucose concentration values which are lower in blood glucose concentration magnitude than their corresponding estimated blood glucose concentration values, thereby decreasing the occurrence of zone D errors.

2. The blood glucose detection system of claim 1, wherein said system has an associated error band and said processing circuit identifies said possible zone D errors as estimated blood glucose values which are greater than or equal to a threshold value of clinical significance and less than the sum of said threshold value and said error band.

3. The blood glucose detection system of claim 2, wherein said error band comprises a maximum expected deviation of said estimated blood glucose concentration values from corresponding actual blood glucose concentration values.

4. The blood glucose detection system of claim 2, wherein said error band comprises a deviation from identity which encompasses a selected percentage of measurements.

5. The blood glucose detection system of claim 4, wherein the selected percentage is at least 80%.

6. The blood glucose detection system of claim 2, wherein said threshold value of clinical significance corresponds to the lowest portion of the border between zone D and zone A of the Clarke error grid.

7. The blood glucose detection system of claim 2, wherein said threshold value of clinical significance is selected from the range of about 50 mg/dL to about 80 mg/dL.

8. The blood glucose detection system of claim 7, wherein said threshold value of clinical significance is about 70 mg/dL.

9. The blood glucose detection system of claim 1, wherein said system has an associated error band and said processing circuit converts said estimated blood glucose concentration values which are identified as possible zone D errors, by subtracting said error band from said estimated blood glucose concentration values which are identified as possible zone D errors.

10. The blood glucose detection system of claim 2, wherein said processing circuit converts said estimated blood glucose concentration values which are identified as possible zone D errors, by subtracting said error band from said estimated blood glucose concentration values which are identified as possible zone D errors.

11. A blood glucose detection system for reducing occurrences of measurement errors that exceed a threshold value of clinical significance, the system having an associated error band, the system comprising:
a processor that converts an estimated blood glucose concentration value of at least the threshold value and less than the sum of the threshold value and the error band into an adjusted blood glucose concentration value that is below the border between zones D and A of the Clarke error grid.

12. The system of claim 11, wherein the threshold value of clinical significance corresponds to the lowest portion of the border between zone D and zone A of the Clarke error grid.

13. The system of claim 11, wherein the error band comprises a maximum expected deviation from actual blood glucose concentration values.

14. The blood glucose detection system of claim 11, wherein said error band comprises a deviation from identity which encompasses a selected percentage of measurements.

15. The blood glucose detection system of claim 14, wherein the selected percentage is at least 80%.

16. An analyte detection system comprising:
a processing circuit; and
a module executable by the processing circuit whereby the processing circuit receives an estimated analyte concentration having an associated first error that is clinically significant, and the processing circuit applies a transfer function to the estimated analyte concentration to generate an adjusted analyte concentration having a second error that is clinically insignificant.

17. The system of claim 16, wherein the first error that is clinically significant comprises an error falling into any of zones C, D, and E of the Clarke error grid.

18. The system of claim 16, wherein the first error that is clinically significant comprises an error falling into any of zones D and E of the Clarke error grid.

19. The system of claim 16, wherein the estimated analyte concentration is an estimate of the concentration of glucose within blood.

20. The system of claim 17, wherein the adjusted analyte concentration differs from the estimated analyte concentration where the estimated analyte concentration has a value from about 70 mg/dL to about 85 mg/dL.

21. The system of claim 16, wherein the system has an associated error band and the adjusted analyte concentration differs from the estimated analyte concentration where the estimated analyte concentration has a value greater than or equal to a threshold value of clinical significance and less than or equal to the sum of the threshold value and the error band.

22. The system of claim 21, wherein the threshold value of clinical significance is selected from the range of about 50 mg/dL to about 80 mg/dL.

23. The system of claim 21, wherein the threshold value of clinical significance is selected from the range of about 60 mg/dL to about 80 mg/dL.

24. The system of claim 21, wherein the threshold value of clinical significance is selected from the range of about 50 mg/dL to about 70 mg/dL.

25. The system of claim 21, wherein the threshold value of clinical significance is selected from the range of about 60 mg/dL to about 70 mg/dL.

26. The system of claim 21, wherein the threshold value of clinical significance corresponds to the lowest portion of a border on the Clarke error grid below which estimated analyte concentrations are zone A errors and above which estimated analyte concentrations are zone D errors.

27. The system of claim 16, wherein the system has an associated error band and the adjusted analyte concentration is about one error band lower than the estimated analyte concentration.

28. The system of claim 16, wherein the system has an associated error band and at least a portion of the transfer function comprises an arc having a radius equivalent to the error band and a center point having horizontal and vertical axis coordinates equal to a threshold value of clinical significance.

29. The system of claim 16, wherein at least a portion of the transfer function comprises an arc.

30. The system of claim 16, wherein the transfer function is continuous.

31. The system of claim 16, wherein the system has an associated error band and the processor adjusts the estimated analyte concentrations having a value greater than or equal to a threshold value of clinical significance and less than or equal to the sum of the threshold value and the error band, to a substantially uniform adjusted value equal to the threshold value.

32. The system of claim 16, wherein the system has an associated error band and the processor adjusts the estimated analyte concentrations having a value greater than or equal to a threshold value of clinical significance and less than or equal to the sum of the threshold value and the error band, to a maximum value less than the threshold value.

33. The system of claim 16, wherein the transfer function is selected to correspond to an individual user.

34. The system of claim 16, wherein the detected analyte is organic.

35. The system of claim 16, wherein the detected analyte is inorganic.

36. The system of claim 16, wherein the analyte is detected from whole blood.

37. The system of claim 16, wherein a plurality of analytes are detected.

38. The system of claim 16, wherein the analyte is detected from tissue.

39. The system of claim 16, wherein the analyte is detected from fluid.

40. The system of claim 16, wherein the analyte is detected from the group consisting of interstitial fluid, intercellular fluid, and whole blood.

41. The system of claim 16, wherein the system is for home use.

42. The system of claim 16, wherein the system is for field use.

43. An apparatus for providing an adjusted analyte concentration, wherein reporting an analyte concentration having a value below a threshold value of clinical significance causes a first course of treatment and reporting an analyte concentration having a value above the threshold value of clinical significance causes a second course of treatment, the apparatus comprising:
a processing circuit that receives an estimated analyte concentration and applies a transfer function to the estimated analyte concentration to provide an adjusted analyte concentration;
wherein the adjusted analyte concentration differs from the estimated analyte concentration when the estimated analyte concentration is in the proximity of the threshold value of clinical significance.

44. The apparatus of claim 43, wherein the estimated analyte concentration is an estimate of the concentration of glucose within blood.

45. The apparatus of claim 43, wherein the adjusted analyte concentration differs from the estimated analyte concentration where the estimated analyte concentration has a value from about 70 mg/dL to about 85 mg/dL.

46. The apparatus of claim 43, wherein estimated analyte concentrations having a value from 70 mg/dL to 85 mg/dL are adjusted.

47. The apparatus of claim 43, wherein the threshold value of clinical significance is selected from the range of about 50 mg/dL to about 80 mg/dL.

48. The apparatus of claim 43, wherein the threshold value of clinical significance is selected from the range of about 60 mg/dL to about 80 mg/dL.

49. The apparatus of claim 43, wherein the threshold value of clinical significance is selected from the range of about 50 mg/dL to about 70 mg/dL.

50. The apparatus of claim 43, wherein the threshold value of clinical significance is selected from the range of about 60 mg/dL to about 70 mg/dL.

51. The apparatus of claim 43, wherein the apparatus has an associated error band and the adjusted analyte concentration differs from the estimated analyte concentration where the estimated analyte concentration has a value greater than or equal to a threshold value of clinical significance and less than or equal to the sum of the threshold value and the error band.

52. The apparatus of claim 51, wherein the threshold value is selected from the range of about 50 mg/dL to about 80 mg/dL.

53. The apparatus of claim 51, wherein the error band is in the range of about 10 mg/dL to about 50 mg/dL.

54. The apparatus of claim 43, wherein the transfer function is continuous.

55. The apparatus of claim 43, wherein the apparatus has an associated error band and the adjusted analyte concentration differs from the estimated analyte concentration by an amount equivalent to the sum of the threshold value and the error band.

56. The apparatus of claim 43, wherein the apparatus has an associated error band and the adjusted analyte concentration differs from the estimated analyte concentration by an amount equivalent to the difference between the estimated analyte concentration and the threshold value.

57. The apparatus of claim 43, wherein the apparatus has an associated error band and the adjusted analyte concentration is selected as the greater of the threshold value and 120% of the difference between the estimated analyte concentration and the error band.

58. The apparatus of claim 43, wherein the apparatus has an associated error band and at least a portion of the transfer function comprises an arc having a radius equivalent to the error band and a center point having horizontal and vertical axis coordinates equal to a threshold value of clinical significance.

59. The apparatus of claim 43, wherein at least a portion of the transfer function comprises an arc segment.

60. The apparatus of claim 43, wherein the adjusted analyte concentration is substantially equivalent to the estimated analyte concentration value when the estimated analyte concentration value is not in the proximity of the threshold value of clinical significance.

61. The apparatus of claim 43, wherein the transfer function is selected to correspond to an individual user.

62. The apparatus of claim 43, wherein the processing circuit reduces a maximum deviation for the estimated analyte concentration.

63. An apparatus for improving the clinical accuracy of an analyte concentration measurement, the apparatus comprising:
detection means for obtaining the analyte concentration measurement;
processor means for adjusting the measurement to avoid reporting erroneous measurements that are clinically significant.

64. A system for determining an analyte concentration, said system comprising a processing circuit which:
computes a first analyte concentration measurement value accurate within a first error band of said system;
determines whether said first analyte concentration measurement value is greater than or equal to a threshold value of clinical significance and less than the sum of said threshold value and said first error band; and
computes a second analyte concentration measurement value when said first analyte concentration measurement value is greater than or equal to said threshold value of clinical significance and less than the sum of said threshold value and said first error band, wherein said second analyte concentration is accurate within a second error band of said system.

65. The system of claim 64, wherein said processor applies a transfer function to obtain an adjusted analyte concentration measurement value when the second analyte concentration measurement value is greater than or equal to said threshold value of clinical significance and less than the sum of said threshold value and said second error band.

66. The system of claim 64, wherein said processor computes said second analyte concentration measurement value by increased sampling of the analyte concentration.

67. The system of claim 64, wherein said processor computes said second analyte concentration measurement value by increasing a sampling time period.

68. A method for improving the clinical accuracy of an analyte concentration measurement, the method comprising:
computing an estimated analyte concentration having an associated first error that is clinically significant; and
processing the estimated analyte concentration to generate an adjusted analyte concentration having a second error that is clinically insignificant.

69. The method of claim 68, wherein the first error that is clinically significant comprises an error falling into any of zones C, D, and E of the Clarke error grid.

70. The method of claim 68, wherein the first error that is clinically significant comprises an error falling into any of zones D and E of the Clarke error grid.

71. The method of claim 68, further comprising determining zones of clinical significance.

72. The method of claim 68, wherein the estimated analyte concentration is an estimate of the concentration of glucose within blood.

73. The method of claim 71, wherein the adjusted analyte concentration differs from the estimated analyte concentration where the estimated analyte concentration has a value from about 70 mg/dL to about 85 mg/dL.

74. The method of claim 68, further comprising determining an error band, wherein the adjusted analyte concentration differs from the estimated analyte concentration where the estimated analyte concentration has a value greater than or equal to a threshold value of clinical significance and less than or equal to the sum of the threshold value and the error band.

75. The method of claim 74, wherein the threshold value of clinical significance is selected from the range of about 50 mg/dL to about 80 mg/dL.

76. The method of claim 74, wherein the threshold value of clinical significance is selected from the range of about 60 mg/dL to about 80 mg/dL.

77. The method of claim 74, wherein the threshold value of clinical significance is selected from the range of about 50 mg/dL to about 70 mg/dL.

78. The method of claim 74, wherein the threshold value of clinical significance is selected from the range of about 60 mg/dL to about 70 mg/dL.

79. The method of claim 74, wherein the threshold value of clinical significance corresponds to the lowest portion of a border on the Clarke error grid below which estimated analyte concentrations are zone A errors and above which estimated analyte concentrations are zone D errors.

80. The method of claim 68, further comprising determining an associated error band, wherein the adjusted analyte concentration is about one error band lower than the estimated analyte concentration.

81. The method of claim 68, further comprising determining an associated error band, wherein the processing adjusts the estimated analyte concentrations having a value greater than or equal to a threshold value of clinical significance and less than or equal to the sum of the threshold value and the error band, to a substantially uniform adjusted value equal to the threshold value.

82. The method of claim 68, further comprising determining an associated error band, wherein the processing adjusts the estimated analyte concentrations having a value greater than or equal to a threshold value of clinical significance and less than or equal to the sum of the threshold value and the error band, to a maximum value less than the threshold value.

83. The method of claim 68, wherein the processing is performed using a transfer function.

84. The method of claim 83, wherein the transfer function is derived from a flow chart.

85. The method of claim 83, wherein the transfer function is derived from a procedural checklist.

86. The method of claim 83, wherein the transfer function is derived from a graph.

87. The method of claim 83, wherein the transfer function is derived from a lookup table.

88. The method of claim 83, further comprising determining an associated error band, wherein at least a portion of the transfer function comprises an arc having a radius equivalent to the error band and a center point having horizontal and vertical axis coordinates equal to a threshold value of clinical significance.

89. The method of claim 83, wherein at least a portion of the transfer function comprises an arc.

90. The method of claim 83, wherein the transfer function is continuous.

91. The method of claim 83, wherein the transfer function is selected to correspond to an individual user.

92. The method of claim 68, wherein the processing is performed using a filter.

93. The method of claim 92, wherein the estimated analyte concentration is a filter input.

94. The method of claim 92, wherein the adjusted analyte concentration provides feedback to the filter.

95. The method of claim 92, wherein the filter is digital.

96. The method of claim 95, wherein a sample period is less than one second.

97. The method of claim 95, wherein a sample period is between one second and one minute.

98. The method of claim 95, wherein a sample period is between one minute and one hour.

99. The method of claim 95, wherein a sample period is greater than one hour.

100. The method of claim 68, wherein the detected analyte is organic.

101. The method of claim 68, wherein the detected analyte is inorganic.

102. The method of claim 68, wherein the analyte is detected from whole blood.

103. The method of claim 68, wherein a plurality of analytes are detected.

104. The method of claim 68, wherein the analyte is detected from tissue.

105. The method of claim 68, wherein the analyte is detected from fluid.

106. The method of claim 68, wherein the analyte is detected from the group consisting of interstitial fluid, intercellular fluid, and whole blood.
